(19) United States
(12) Patent Application Publication
(10) Pub. No.: US 2023/0050339 A1
(43) Pub. Date: Feb. 16, 2023

(54) COMPOSITIONS AND METHODS FOR TREATING LIVER DISEASE

(71) Applicant: Children's Hospital Medical Center, Cincinnati, OH (US)

(72) Inventor: Pranav-Kumar Shivakumar, Cincinnati, OH (US)

(21) Appl. No.: 17/796,796

(22) PCT Filed: Feb. 17, 2021
(86) PCT No.: PCT/US21/18326

§ 371 Date: Aug. 1, 2022

Related U.S. Application Data
(60) Provisional application No. 62/977,770, filed on Feb. 18, 2020.

(51) Int. Cl. C07K 16/18; A61K 31/19; C07K 16/40; A61P 1/16; A61P 29/00

(52) U.S. Cl. CPC C07K 16/18; A61K 31/19; C07K 16/40; A61P 1/16; A61P 29/00; C07K 2317/76; A61K 2039/505

(57) ABSTRACT

Disclosed are methods of treating an individual, for example, a human individual, or more particularly a pediatric individual, having a disease characterized by liver fibrosis, comprising administering a therapeutically effective amount of one or both of a Complement Factor B inhibitor and a Complement Factor P (“CFP” or “properdin”) inhibitor, e.g., anti-CFP antibody and/or anti-CFB antibody or the respective antigen-binding fragment(s) thereof. Exemplary disease states include, but are not limited to, biliary atresia and post-Kasai biliary atresia.

### CROSS REFERENCE TO RELATED APPLICATIONS

This application claims the benefit of, and priority to, U.S. Provisional Application Ser. No. 62/977,770, filed Feb. 18, 2020, the contents of which are incorporated by reference in its entirety for all purposes.

### STATEMENT REGARDING FEDERALLY-SPONSORED RESEARCH

This invention was made with government support under DK078392 awarded by the National Institutes of Health. The government has certain rights in the invention.

### BACKGROUND

Liver disease and conditions associated with liver disease, such as liver fibrosis, is a problem in the art for which new treatments are needed. Liver fibrosis may occur when repetitive or long-lasting injury or inflammation causes excessive amounts of scar tissue to build up in the organ, a condition that can result from most types of chronic liver disease.

In particular, pediatric cholestatic liver diseases affect a small percentage of children, but therapy results in significant healthcare costs each year. Currently, many of the pediatric cholestatic liver diseases require invasive and costly treatments such as liver transplantation and surgery. An effective and less invasive treatment that is suitable for the pediatric population is not available. Biliary Atresia (BA), in particular, is a pediatric liver disease restricted to newborn infants with no known medical treatment. BA is a rare neonatal disease manifesting only in the first few weeks of life characterized by ascending obstruction of the biliary tree resulting in severe cholestasis and rapidly progressing biliary cirrhosis. The common histopathological picture is one of inflammatory damage to the intra- and extrahepatic bile ducts with sclerosis and narrowing or obliteration of the biliary tree. BA is a rapidly progressing obliterative disease of the extra- and intra-hepatic bile ducts and represents an extreme spectrum of neonatal cholestasis. Children who develop BA are born jaundice-free; however, within the first weeks of life, the extrahepatic biliary tree develops inflammation leading to duct obstruction and loss of bile flow.

Untreated, this condition leads to cirrhosis and death within the first years of life. BA remains the most common indication for pediatric liver transplantation worldwide. The incidence of B.A is approximately 1:10-15,000 of live births and is classified as a rare disease by National Organization of Rare Disorders (NORD) and NIDDK (National Institute of Diabetes and Digestive and Kidney Diseases. Children who develop BA are born jaundice-free; however, within the first weeks of life, the extrahepatic biliary tree develops inflammation leading to duct obstruction and loss of bile flow. The baby suffers from acholic (chalk-colored) stools, yellowing of skin, enlarged liver and spleen, ascites develops with rapidly progressing liver injury and cirrhosis, and the baby suffers from loss of weight, becomes irritable and has worsening jaundice. Infants with BA are severely ill and may face developmental challenges even after liver transplantation. Infants affected by BA represent an extreme spectrum of neonatal cholestasis and show progressive jaundice and growth retardation. Because of the severe clinical manifestations and limited therapeutic options, most infants progress to end-stage liver cirrhosis, portal hypertension and liver failure eventually needing liver transplantation.

Intraoperative cholangiogram is the only mechanism available for a definitive diagnosis of BA. Because of the progressive nature of the disease, infants, at the time of diagnosis present with a scarred extrahepatic bile duct with varying degrees of intrahepatic inflammation and fibrosis. Surgical intervention by Kasai portoenterostomy (KPE) is the only treatment option, which removes the entire fibrosed biliary tree and surgically recreates an intestinal anastomosis to establish bile flow. While the postsurgical medical management combines nutrition, antibiotics, choleretics, and possibly anti-inflammatory medications, the impact of these practices on the clinical outcome is unclear, and there are no medical therapies available to prevent or reduce the likelihood of ongoing liver injury following a Kasai procedure. Further, infants having a “failed Kasai” will require a liver transplant in infancy to survive, and infants diagnosed too late have too much liver damage to benefit from Kasai and will require early transplant. These two groups of patients encompass about ⅓ to more than half of the BA population. In fact, the Kasai procedure only restores bile flow and 80% of patients still progress to failure.

Post-operative complications are also significant in that some patients, even after successful bile drainage, can still experience cholangitis and succumb to infection. Despite the clinical success of resolving extrahepatic bile duct manifestations of the disease, progression of the liver disease involving intrahepatic bile ducts continue in a majority of children resulting in cirrhosis, with only 13-50% of patients alive with native liver by 2 years of age. In infants progressing to end-stage cirrhosis, liver transplantation is the only option—assuming an average cost of $200-300K per transplant, the economic burden to treat children with BA is approximately—$134 million annually placing significant strain on the affected families as well as on health-care resources and service utilization costs. This is further compounded by a complete lack of medical interventions.

The current nontransplant treatment strategies are at best palliative and primarily make use of steroids in the immediate post-operative period due to their anti-inflammatory properties. However, the role of corticosteroids in improving bile flow is controversial. Indeed, several clinical trials including the most recent and extensive trial of corticosteroid therapy in the US following Kasai (ChiLDREN; START trial: NCT00294684) showed that steroids alone do not prevent the need for liver transplantation. Outcomes from most of these trials strongly suggest the existence of inflammatory footprints beyond the immune-suppressive capacity of, or pathways regulated by steroids. Of significance, in the current pediatric end-stage liver disease (PELD) system, children with BA face the risk of not receiving a liver in a safe and timely manner.

Thus, identification of effective treatments for hepatobiliary disease states is of importance. The instant disclosure seeks to address one or more of the aforementioned needs in the art.

### BRIEF SUMMARY

Disclosed are compositions and methods for the treatment of an individual having liver fibrosis or a disease state characterized by liver fibrosis, via administration of a therapeutically effective amount of an inhibitor of alternative/amplification pathway of complement, for example, of one or both of a Complement Factor B inhibitor and a Complement Factor P (“CFP” or “properdin”) inhibitor.

In some embodiments, the disclosure relates to an inhibitor of the disclosure, e.g., anti-CFP antibody and/or anti-CFB antibody or the respective antigen-binding fragment(s) thereof, for use in treatment of a disease characterized by liver fibrosis, e.g. BA. In some embodiments, the disclosure relates to use of an inhibitor of the disclosure, e.g., anti-CFP antibody and/or anti-CFB antibody or the respective antigen-binding fragment(s) thereof (or a composition comprising the same), for the manufacture of a medicament for the treatment of a disease characterized by liver fibrosis, e.g. BA.

In some embodiments, the compositions and methods described herein are useful in the treatment of biliary atresia. Biliary atresia is a rare disease of the liver and bile ducts that occurs in infants and toddlers. Symptoms of the disease typically appear or develop about two to eight weeks after birth. Cells within the liver produce liquid called bile, which helps to digest fat and waste products from the liver to the intestines for excretion. This network of channels and ducts (biliary system) allows drainage of bile from the liver into the intestines.

In biliary atresia, bile flow from the liver to the gallbladder is blocked, causing the bile to be trapped inside the liver. This causes damage and scarring of the liver cells (cirrhosis) and eventually liver failure.

Symptoms of biliary atresia typically appear within the first two weeks to two months of life. Symptoms include, but are not limited to, jaundice, dark urine, acholic stools (clay-colored stools), weight loss and irritability. In some embodiments, BA manifests together with heart disorder; spleen disorder (e.g., polysplenia); disorder of blood vessels (e.g., inferior vena caval anomalies, preduodenal portal vein); intestinal disorder (e.g., situs inversus or malrotation). In cases of BA that is treated using surgery, symptoms of disease include, bruising of the skin, nosebleeds, retention of body fluid and enlarged veins (varices) in the stomach and esophagus. In some embodiments, increased pressure in these veins can cause a sudden and large amount of bleeding in the stomach and intestines.

Diagnosis of BA may be carried out using, e.g., blood tests for liver function abnormalities; X-rays of the abdomen to identify enlarged liver and spleen; an abdominal ultrasound to diagnose small gall bladder or absence of gall bladder. In some embodiments, diagnosis is made using liver biopsy, comprising, removal of a tiny sample of the liver (with a needle) and analysis thereof. In some embodiments, diagnosis is made surgically. Surgery for BA allows doctors to identify injured bile ducts and/or other abnormalities of the biliary system. In some embodiments, BA is diagnosed with an operative cholangiogram.

In some embodiments, the subject (or patient) with BA may be treated prior to therapy with the compositions and/or kits described herein. For instance, a Kasai procedure or hepatoportoenterostomy may have been performed in the patients. The Kasai procedure is an operation to re-establish bile flow from the liver into the intestine, wherein the damaged ducts outside of the liver (extrahepatic ducts) are removed and smaller ducts that are still open and draining bile are identified. Then, a loop of intestine is surgically attached to this portion of the liver, so that bile can flow directly from the remaining healthy bile ducts into the intestine. Kasai procedure is accompanied by long-term antibiotic therapy, and additional medications may be used to promote bile flow and maximize the success of the operation

Since long-term survival after the Kasai procedure is affected by the presence of progressive liver disease (cirrhosis) and the development of portal hypertension (high blood pressure in the portal vein that carries blood to the liver), the subject (or patient) may undergo subsequent tests (post-surgery) to minimize complications due to cirrhosis and/or portal hypertension.

The present application discloses a hitherto unrecognized involvement of the complement alternative pathway, for example, complement factor P and complement factor B, in biliary atresia. Preliminary studies on co-localization of complement pathway components in in vivo model of BA showed that C3b signals localize to peribiliary gland in normal extrahepatic bile duct and expand upon RRV infection with increased C3b content, with C3b signals localizing to injured duct epithelium and areas of active wound during inflammation and on atretic duct at the terminal stages. C5b9 concurrently co-localized with C3b, spreading from apical region of cholangiocytes to injured regions to immune cell infiltrates with time. In human BA patients, the levels of serum C3b and iC3b levels and C3a and C3adesArg levels were increased; levels of CFD, CFH and/or CFI; along with levels of CFHR5, CD55 and/or C9, were differentially expressed. Additionally, several mediators of complement pathway were found to be enhanced in BA animal models and patients compared to controls.

In the therapeutic context, antibody-mediated blockage of CFP attenuated various signs and/or symptoms of BA. For instance, in animal models of BA, treatment with an anti-CFP antibody (a) prevented acute growth failure and promoted weight gain in neonatal mice; (b) promoted recovery from jaundice associated with development of BA; (c) prevented early mortality in a significant percentage (e.g., >about 85%) of population; (d) prevented EHBD inflammation, cholangiocyte injury and progression to duct atresia; (e) attenuated ductal edema, mucosal immune cell infiltrations and epithelial disruptions; (f) prevented portal inflammation, loss of hepatocytes and necroinflammatory patches; (g) attenuated liver portal inflammation and necroinflammatory changes characteristic of neonatal BA; (h) suppressed reactive bile duct proliferation (which serves as a poor prognostic factor in patients with BA); (i) attenuated intrahepatic bile duct proliferations; (j) substantially decreased immune cell infiltrations, portal inflammation and hepatocellular necrosis; (j) attenuated portal inflammation, liver injury and necrosis and intrahepatic cholangitis; (k) reduced portal biliary fibrosis in infantile mice with liver fibrosis; (l) attenuated biliary fibrosis and portal expansions characteristic of progressive BA; (m) substantially suppressed bile duct proliferations associated with progressive hepatobiliary disease; and/or (n) attenuated intrahepatic bile duct proliferations in infantile mice with BA-associated liver fibrosis.

In a second therapeutic context, antibody-mediated blockage of CFB attenuated various signs and/or symptoms of BA. For instance, in animal models of BA, treatment with an anti-CFB antibody a) prevented acute growth failure and promoted weight gain; b) promoted recovery from jaundice associated with development of BA; c) prevented early mortality in a significant % of the animal population (e.g., >about 85%) with experimental BA; d) prevented extrahepatic bile duct (EHBD) inflammation, cholangiocyte injury and progression to duct atresia; e) attenuated ductal edema, mucosal immune cell infiltrations and epithelial disruptions; f) prevented portal inflammation, loss of hepatocytes and development of necroinflammatory patches; g) attenuated liver portal inflammation and necroinflammatory changes characteristic of neonatal BA; h) suppressed reactive bile duct proliferation (a poor prognostic factor BA patients); i) attenuated intrahepatic bile duct proliferations; j) substantially decreased immune cell infiltrations, portal inflammation and hepatocellular necrosis; k) attenuated portal inflammation, liver injury and necrosis and intrahepatic cholangitis; l) significantly reduced portal biliary fibrosis in infantile mice with liver fibrosis; m) attenuates biliary fibrosis and portal expansions characteristic of progressive BA; n) substantially suppressed bile duct proliferations associated with progressive hepatobiliary disease; and/or o) attenuated intrahepatic bile duct proliferations in infantile mice with BA-associated liver fibrosis.

These data establish the effectiveness of compositions comprising antagonists of the complement alternative pathway, such as anti-CFP antibodies and/or anti-CFB antibodies, or a combination thereof (e.g., a composition containing anti-CFP antibodies and anti-CFB antibodies) for the treatment of biliary atresia and various related diseases, e.g., cirrhosis.

The disclosure accordingly relates to the following non-limiting embodiments:

In some embodiments, the disclosure relates to a method for treating a disease characterized by biliary fibrosis, comprising administering a therapeutically effective amount of one or both of a Complement Factor B inhibitor and a Complement Factor P (“CFP” or “properdin”) inhibitor to an individual or subject having the disease. Particularly, the disclosure relates to method(s) for treating a disease which is cholestatic liver disease or biliary atresia (BA). Especially, the disclosure relates to method(s) for treating post-Kasai biliary atresia.

In some embodiments, the disclosure relates to method(s) for treating a disease characterized by biliary fibrosis, comprising administering a therapeutically effective amount of the CFB inhibitor or the CFP inhibitor, which is an antibody, e.g., anti-CFP antibody and/or anti-CFB antibody.

In some embodiments, the disclosure relates to method(s) for treating a disease characterized by biliary fibrosis, comprising administering a therapeutically effective amount of a whole antibody or an antibody fragment, e.g., an antigen-binding antibody fragment, wherein the antibody is anti-CFP antibody and/or anti-CFB antibody.

In some embodiments, the disclosure relates to method(s) for treating a disease characterized by biliary fibrosis, comprising administering an inhibitor which is a human, humanized, chimerized, or deimmunized antibody or antibody fragment, e.g., anti-CFP antibody and/or anti-CFB antibody or the respective antigen-binding fragment thereof.

In some embodiments, the disclosure relates to method(s) for treating a disease characterized by biliary fibrosis in a human subject comprising implementing any of the foregoing or following embodiments. The individual or the subject may be a pediatric subject.

In some embodiments, the disclosure relates to method(s) for treating a disease characterized by biliary fibrosis in a subject comprising administering the CFB inhibitor and/or CFP inhibitor for a period of time sufficient to attenuate and/or reverse liver fibrosis. Particularly, the inhibitor is administered in an amount and for a period of time sufficient to attenuate and/or reverse one or more of biliary-atresia associated hepatobiliary injury, intrahepatic and/or hepatobiliary inflammation, intrahepatic and/or hepatobiliary fibrosis, cholangiopathy, periductal inflammation, fibrosis, ballooning degeneration, confluent necrosis, portal inflammation, lobular inflammation, bile duct injury, bile duct fibrosis, portal and and/or pericellular bile duct fibrosis, and combinations thereof, in the subject.

In some embodiments, the disclosure relates to method(s) for treating a disease characterized by biliary fibrosis in a subject comprising administering the CFB inhibitor and/or CFP inhibitor for a period of time sufficient to promote a regenerative response in a liver and/or a bile duct cell in the subject. Particularly, the administration of the inhibitor preserves, restores, or improves liver function in the subject. Still particularly, the administration of the inhibitor reduces the need for a liver transplant in the subject.

In some embodiments, the disclosure relates to method(s) for treating a disease characterized by biliary fibrosis, comprising administering a therapeutically effective amount of an CFB inhibitor or an CFP inhibitor or both inhibitors to an individual or subject, wherein the administration occurs after the individual or the subject has undergone a Kasai procedure, for example, wherein a first dose is administered at a time point selected from at the time of the Kasai procedure, within about 1 to 72 hours of the Kasai procedure, or within about 8 to 36 hours of the Kasai procedure, or within about 48 hours of the Kasai procedure.

In some embodiments, the disclosure relates to method(s) for treating a disease characterized by biliary fibrosis, comprising administering a therapeutically effective amount of an CFB inhibitor or an CFP inhibitor or both inhibitors to an individual or subject, wherein the inhibitor is administered in an amount sufficient to improve or substantially normalize serum biomarkers of liver injury selected from one or more of conjugated bilirubin (Bc), aspartate aminotransferase (AST), alanine aminotransferase (ALT), gamma-glutamyltransferase (GGT), albumin, sodium, total bilirubin (TB), platelets, international normalized ratio (INR), 25-hydroxy Vitamin D, Vitamin A, and Vitamin E.

In some embodiments, the disclosure relates to method(s) for treating a disease characterized by biliary fibrosis, comprising administering a therapeutically effective amount of an CFB inhibitor or an CFP inhibitor or both inhibitors to an individual or subject, wherein the inhibitor is administered until an outcome of one or more parameters is achieved: normalization or improvement of Total serum bile acids (TSBAs), an improvement or normalization of weight gain, an improvement or normalization of total bilirubin concentration; an improvement or normalization of weight height; an improvement or normalization of ascites, an improvement or normalization of bile drainage, an improvement or normalization of circulating Tregs (CD4+CD25+FoxP3+), CD3/4 T cells, CD3/8 T cells, NK cells (CD56), NK T cells (CD3/56), CD19/20 B cells, macrophages (CD14/11b), and neutrophils, an improvement or normalization of plasma levels of anti-enolase antibody, an improvement or normalization of plasma cytokine levels (Th1/Th2 multiplex and IL17), and improvement or reversal of inflammation and/or fibrosis progression.

In some embodiments, the disclosure relates to method(s) for treating a disease characterized by biliary fibrosis as provided in the foregoing or following embodiments, wherein the CFB inhibitor or CFP inhibitor or both is administered intravenously and/or subcutaneously. In one aspect, the CFB inhibitor or the CFP inhibitor is administered daily, or at least every other day, or at least twice a week, or at least weekly, or at least bi-weekly, or at least once a month.

In some embodiments, the disclosure relates to method(s) for treating a disease characterized by biliary fibrosis as provided in the foregoing or following embodiments, wherein the CFB inhibitor or CFP inhibitor or both is administered together with N-acetyl cysteine (NAC).

In some embodiments, the disclosure relates to a composition comprising a Complement Factor B inhibitor and a Complement Factor P (“CFP” or “properdin”) inhibitor, or a combination thereof for use in the treatment of a disease characterized by or leading to biliary fibrosis in an individual or a subject. In some embodiments, the composition further comprises N-acetyl cysteine.

In some embodiments, the disclosure relates to a composition comprising a CFB inhibitor or a CFP inhibitor, or a combination thereof, optionally together with N-acetyl cysteine, wherein the composition attenuates and/or reverses one or more signs or symptoms of biliary-atresia associated hepatobiliary injury selected from intrahepatic and/or hepatobiliary inflammation, intrahepatic and/or hepatobiliary fibrosis, cholangiopathy, periductal inflammation, fibrosis, ballooning degeneration, confluent necrosis, portal inflammation, lobular inflammation, bile duct injury, bile duct fibrosis, portal and and/or pericellular bile duct fibrosis, and combinations thereof, in an individual or a subject receiving the composition.

In some embodiments, the disclosure relates to a composition comprising a CFB inhibitor or a CFP inhibitor, or a combination thereof, optionally together with N-acetyl cysteine, wherein the composition promotes a regenerative response in a liver and/or a bile duct cell in an individual or a subject receiving the composition.

In some embodiments, the disclosure relates to a composition comprising a CFB inhibitor or a CFP inhibitor, or a combination thereof, optionally together with N-acetyl cysteine, wherein the composition preserves, restores, or improves liver function in an individual or a subject receiving the composition.

In some embodiments, the disclosure relates to a composition comprising a CFB inhibitor or a CFP inhibitor, or a combination thereof, optionally together with N-acetyl cysteine, wherein the composition reduces the need for a liver transplant in an individual or a subject.

In some embodiments, the disclosure relates to a composition comprising a CFB inhibitor or a CFP inhibitor, or a combination thereof, optionally together with N-acetyl cysteine, wherein the composition comprises a pharmaceutically acceptable excipient or pharmaceutically acceptable carrier.

### BRIEF DESCRIPTION OF THE DRAWINGS

This application file may contain at least one drawing executed in color. Copies of this patent or patent application publication with color drawing(s) will be provided by the Office upon request and payment of the necessary fee.

The details of one or more embodiments of the disclosure are set forth in the accompanying drawings/tables and the description below. Other features, objects, and advantages of the disclosure will be apparent from the drawings/tables and detailed description, and from the claims.

FIG. 1 shows C3b signals localize to peribiliary gland (PBGs) in normal extrahepatic bile duct (EHBD) and expand upon RRV infection with increased C3b content.

FIG. 2 shows during inflammation, C3b signals localize to injured duct epithelium and areas of active wound.

FIG. 3 shows at terminal stages, C3b deposits on atretic duct comprises mostly of necrotic and fibrous tissue.

FIG. 4 shows quantification of EHBD immunofluorescent signals for C3b deposits shows differential expressions. Statistical significance determined between saline and RRV groups at each timepoint using nonparametric unpaired t Test with Welch's correction. P<0.0001 at all time-points.

FIG. 5 shows immunostaining in livers from mice with BA shows intense deposition of C3b to portal vein endothelial cells and to areas of hepatocyte necrosis. In patients with BA, vascular remodeling, deposition of C3b and activation of alternate pathway (AP) may contribute to disappearance of interlobular bile ducts.

FIG. 6 shows concurrent localization of C5b9 (with C3b) to apical region of cholangiocytes during early duct injury (day 3).

FIG. 7 shows at day 7, C5b9 signals localize to injured and apical region of cholangiocytes and immune cell infiltrates.

FIG. 8 shows at day 14, C5b9 signals localize to injured cholangiocytes and immune cell infiltrates.

FIG. 9 shows quantification of EHBD immunofluorescent signals for C5b9 deposits showing differential expression at various time points. Statistical significance determined between saline and RRV groups at each timepoint using nonparametric unpaired t Test with Welch's correction. P<0.0001 at all time-points.

FIG. 10 shows bar charts of SOMALOGIC SOMASCAN of serum C3b and iC3b levels (as assessed using SOMALOGIC SOMASCAN), showing increased C3b and iC3b levels in patients with BA (**** indicates statistical significance; P<0.0001).

FIG. 11 shows bar charts of serum C3a and C3adesArg levels (as assessed using SOMALOGIC SOMASCAN), showing increased C3a and C3adesArg levels in patients with BA (**** indicates statistical significance; P<0.0001).

FIG. 12 shows bar charts of serum CFD, CFH and CFI levels (as assessed using SOMALOGIC SOMASCAN), showing differential expression of these proteomic markers in patients with BA (* indicates statistical significance; P<0.05).

FIG. 13 shows bar charts of serum CFHR5, CD55 and C9 levels (as assessed using SOMALOGIC SOMASCAN), showing differential expression of these proteomic markers in patients with BA (** indicates statistical significance; P<0.001).

FIG. 14 shows bar charts of normalized expression levels of the inhibitory factor CFI in normal subjects versus patients with BA. CFI levels were measured using RNASeq analysis of livers from patients with BA. CFI serves as a potent inactivator of C3b and plays an important role in regulating and inhibiting the immune response by controlling all complement pathways. Important co-factors include CFH, CD46 (cell surface) and C4BP (fluid phase) (** indicates statistical significance; P<0.001).

FIG. 15 shows bar charts of normalized expression levels of the key regulatory factor CFH in normal subjects versus patients with BA. CFH levels were measured using RNASeq analysis of livers from patients with BA. CFH acts as a soluble inhibitor of complement and accelerates the decay of the complement alternative pathway (AP) C3 convertase C3bBb, thus preventing local formation and amplification of additional C3b (**** indicates statistical significance; P<0.0001).

FIG. 16 shows bar charts of normalized expression levels of the key regulatory factor CD46 in normal subjects versus patients with BA. CD46 levels were measured using RNASeq analysis of livers from patients with BA. CD46 acts as a cofactor for CFI and protects autologous cells against complement mediated injury by cleaving C3b and C4b deposited on host tissue. Also acts as a costimulatory factor for T-cells which induces the differentiation of CD4+ into regulatory T-cells (T-regs) (** indicates statistical significance; P<0.001).

FIG. 17 shows bar charts of normalized expression levels of C4BPA and C4BPB in normal versus patients with BA. Livers from patients with BA showed significantly lower expressions of both C4BPA and C4BPB (**** indicates statistical significance; P<0.0001).

FIG. 18 shows bar charts of normalized expression levels of the activating factor CFD in normal subjects versus patients with BA. CFD levels were measured using RNASeq analysis of livers from patients with BA. CFD is known to cleave CFB as its only substrate when complexed to C3b and is the rate-limiting step of AP of complement activation. Increased levels are found in heart failure patients with advanced clinical disease (**** indicates statistical significance; P<0.0001).

FIG. 19 shows bar charts of key complement components C9, CD59 and CFHR5 levels (as assessed using RNASeq analysis), showing differential expression of these markers in patients with BA (** indicates statistical significance at P<0.01; *** indicates statistical significance at P<0.0005).

FIG. 20 shows CFP levels in extrahepatic bile duct (EHBD; left panel) and liver (right panel) are increased in experimental BA correlating to hepatobiliary inflammation and fibrosis. Here, RNASeq data from EHBDs and livers were analyzed for expression of CFP and validated by real-time qPCR estimations and normalized to GAPDH levels (* indicates statistical significance at P<0.05; ** indicates statistical significance at P<0.01; *** indicates statistical significance at P<0.0005; **** indicates statistical significance; P<0.0001).

FIG. 21 shows CFP levels in serum of patients with BA. Serum CFP levels were estimated using high throughput SOMALOGIC SOMASCAN proteomics in BA patients (N=137) and normal subjects (N=7). Decreased levels of CFP are associated with advanced clinical disorder and clear manifestations of the disease due to consumption. Specifically, consumption of CFP indicates activation of alternate pathway (AP) (*** indicates statistical significance at P<0.0005).

FIG. 22A and FIG. 22B show CFP expressions in livers of patients with BA, showing patterns of attenuated CFP levels and increased consumption in BA patients.

FIG. 23 shows that blocking CFP prevents acute growth failure and promotes weight gain in neonatal mice with experimental BA.

FIG. 24 shows anti-CFP antibodies (Abs) promote recovery from jaundice associated with development of BA in early neonatal life.

FIG. 25 shows treatment with anti-CFP Abs prevents early mortality in >about 85% of mice with experimental BA.

FIG. 26A, FIG. 26B and FIG. 26C show that blocking CFP prevents EHBD inflammation, cholangiocyte injury and importantly progression to duct atresia. Histology of EHBDs after anti-CFP Ab treatments show significant reductions in epithelial injury, periductal inflammation and intra-ductal immune cell infiltrations (magnification: about 400×).

FIG. 27 shows blocking CFP attenuates ductal edema, mucosal immune cell infiltrations and epithelial disruptions. IMAGEJ quantifications were performed using 5 μm thick H&E stained sections from EHBDs obtained from RRV-infected, Buffer and anti-CFP Ab treated neonatal mice at day 14 post infection (* indicates statistical significance at P<0.05; **** indicates statistical significance; P<0.0001).

FIG. 28A and FIG. 28B shows anti-CFP Ab treatment prevents portal inflammation, loss of hepatocytes and necroinflammatory patches.

FIG. 29 shows that blocking CFP attenuates liver portal inflammation and necroinflammatory changes characteristic of neonatal BA (** indicates statistical significance at P<0.01; **** indicates statistical significance at P<0.0001).

FIG. 30A and FIG. 30B show that anti-CFP Ab treatment suppressed reactive bile duct proliferation, a poor prognostic factor in patients with BA.

FIG. 31 shows blocking CFP attenuates intrahepatic bile duct proliferations in neonatal mice with experimental BA. IMAGEJ quantifications were performed using 5 μm thick sections from livers of RRV-infected, Buffer and anti-CFP Ab treated mice at day 14 post infection and stained using pan-cytokeratin antibodies (**** indicates statistical significance at P<0.0001).

FIG. 32A and FIG. 32B show that blocking CFP substantially decreased immune cell infiltrations, portal inflammation and hepatocellular necrosis.

FIG. 33 shows blocking CFP attenuates portal inflammation, liver injury and necrosis and intrahepatic cholangitis. IMAGEJ quantifications were performed using 5 μm thick H&E stained liver sections from RRV-infected, Buffer and anti-CFP Ab treated neonatal mice at day 19 post infection (** indicates statistical significance at P<0.01; **** indicates statistical significance at P<0.0001).

FIG. 34A and FIG. 34B show that treatment with anti-CFP Abs significantly reduced portal biliary fibrosis in infantile mice with liver fibrosis.

FIG. 35 shows blocking CFP attenuates biliary fibrosis and portal expansions characteristic of progressive BA. IMAGEJ quantifications were performed using 5 μm thick Sirius Red stained liver sections of RRV-infected, buffer and antibody-CFP Ab treated infantile mice at day 19 post infection (**** indicates statistical significance at P<0.0001).

FIG. 36A and FIG. 36B show that anti-CFP Abs substantially suppressed bile duct proliferations associated with progressive hepatobiliary disease.

FIG. 37 shows that blocking CFP attenuates intrahepatic bile duct proliferations in infantile mice with BA-associated liver fibrosis. IMAGEJ quantifications were performed using 5 μm thick liver sections from RRV-infected, buffer and anti-CFP Ab treated mice at day 19 post infection and stained using pan-cytokeratin antibodies (*** indicates statistical significance at P<0.0005).

FIG. 38A and FIG. 38B show that complement factor B (CFB) is increased in experimental biliary atresia (BA) correlating to hepatobiliary inflammation and fibrosis RNASeq data from EHBDs and livers were analyzed for expression of CFB and validated by real-time qPCR estimations and normalized to GAPDH levels (* indicates statistical significance at P<0.05; ** indicates statistical significance at P<0.01; **** indicates statistical significance at P<0.0001).

FIG. 39 shows that CFB levels in serum of patients with BA: Increased CFB levels and stratification into high and low expressors. Serum CFB levels were estimated using high throughput SOMALOGIC SOMASCAN proteomics in BA patients (N=137) and normal subjects (N=7). Increased levels of CFB indicate systemic dysregulation of complement activation and advanced clinical disorder disease (** indicates statistical significance at P<0.01).

FIG. 40A and FIG. 40B show livers of patients with BA show high expressions of CFB localized to epithelial and immune cells in portal areas.

FIG. 41 shows that blocking CFB prevents acute growth failure and promotes weight gain in neonatal mice with experimental BA.

FIG. 42 shows anti-CFB antibodies (Abs) promote recovery from jaundice associated with development of BA in early neonatal life.

FIG. 43 shows treatment with anti-CFB Abs prevents early mortality in >about 85% of mice with experimental BA.

FIG. 44A, FIG. 44B, FIG. 44C, FIG. 44D, and FIG. 44E show blocking CFB prevents extrahepatic bile duct (EHBD) inflammation, cholangiocyte injury and importantly progression to duct atresia. Histology of EHBDs after anti-CFB Ab treatments show significant reductions in epithelial injury, periductal inflammation and intra-ductal immune cell infiltrations (magnification: 400×).

FIG. 45 shows that blocking CFB attenuates ductal edema, mucosal immune cell infiltrations and epithelial disruptions. IMAGEJ quantifications were performed using 5 μm thick H&E stained sections from EHBDs obtained from RRV-infected, buffer and anti-CFB Ab treated neonatal mice at day 14 post infection (*** indicates statistical significance at P<0.005; **** indicates statistical significance at P<0.0001).

FIG. 46A and FIG. 46B show anti-CFB Ab treatment prevents portal inflammation, loss of hepatocytes and necroinflammatory patches.

FIG. 47 shows that blocking CFB attenuates liver portal inflammation and necroinflammatory changes characteristic of neonatal BA. IMAGEJ quantifications were performed using 5 μm thick H&E stained liver sections of RRV-infected, buffer and anti-CFB Ab treated neonatal mice at day 14 post infection (**** indicates statistical significance at P<0.0001).

FIG. 48A and FIG. 48B show that anti-CFB Ab treatment suppressed reactive bile duct proliferation, a poor prognostic factor in patients with BA.

FIG. 49 shows that blocking CFB attenuates intrahepatic bile duct proliferations in neonatal mice with experimental BA. IMAGEJ quantifications were performed using 5 μm thick sections from livers of RRV-infected, buffer and anti-CFB Ab treated mice at day 14 post infection and stained using pan-cytokeratin antibodies (**** indicates statistical significance at P<0.0001).

FIG. 50A and FIG. 50B show that blocking CFB substantially decreased immune cell infiltrations, portal inflammation and hepatocellular necrosis.

FIG. 51 shows that blocking CFB attenuates portal inflammation, liver injury and necrosis and intrahepatic cholangitis. IMAGEJ quantifications were performed using 5 μm thick H&E stained liver sections from RRV-infected, buffer and anti-CFB Ab treated neonatal mice at day 19 post infection (** indicates statistical significance at P<0.001; **** indicates statistical significance at P<0.0001).

FIG. 52A and FIG. 52B show that treatment with anti-CFB Abs significantly reduced portal biliary fibrosis in infantile mice with liver fibrosis.

FIG. 53 shows that blocking CFB attenuates biliary fibrosis and portal expansions characteristic of progressive BA. IMAGEJ quantifications were performed using 5 μm thick Sirius Red stained liver sections of RRV-infected, buffer and anti-CFB Ab treated infantile mice at day 19 post infection (**** indicates statistical significance at P<0.0001).

FIG. 54A and FIG. 54B show anti-CFB Abs substantially suppressed bile duct proliferations associated with progressive hepatobiliary disease.

FIG. 55 shows that blocking CFB attenuates intrahepatic bile duct proliferations in infantile mice with BA-associated liver fibrosis. IMAGEJ quantifications were performed using 5 μm thick liver sections from RRV-infected, buffer and anti-CFB Ab treated mice at day 19 post infection and stained using pan-cytokeratin antibodies. (**** indicates statistical significance at P<0.0001).

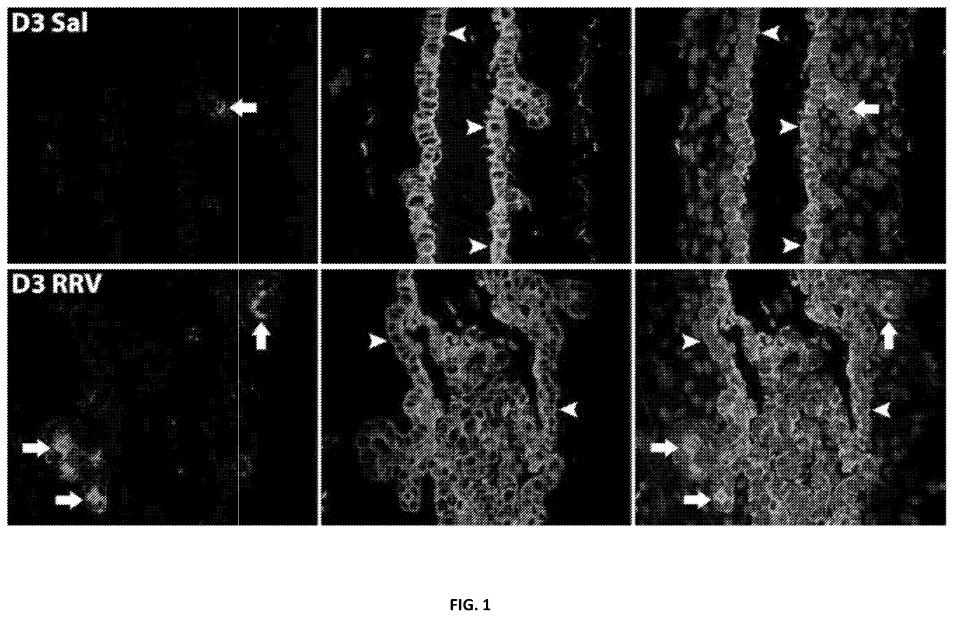

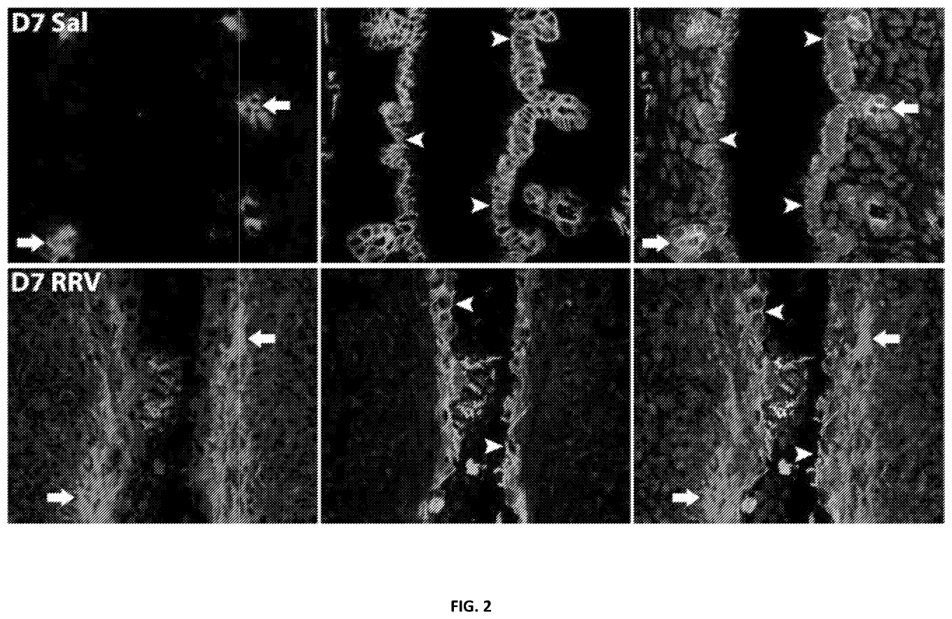

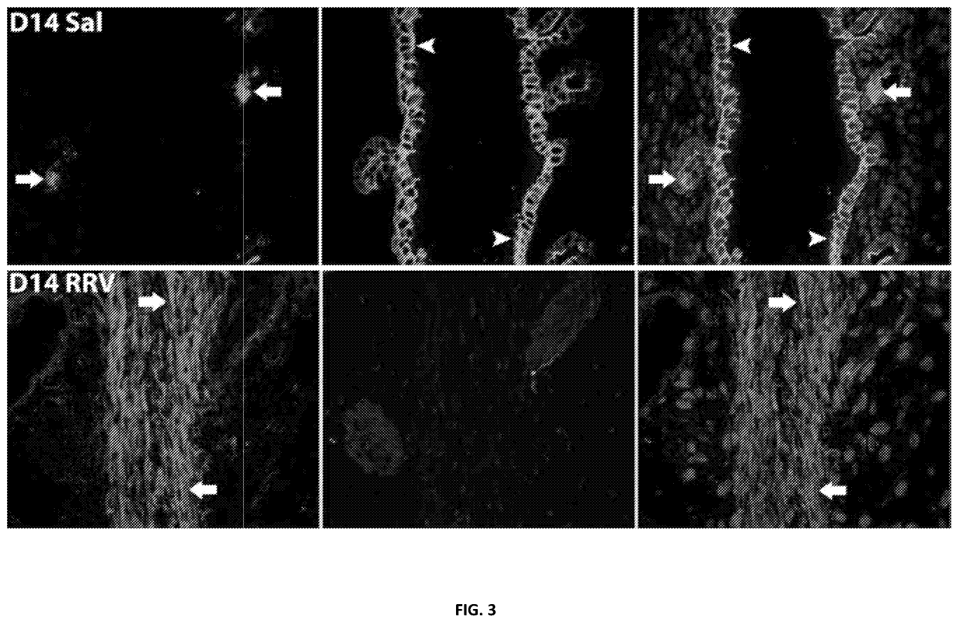

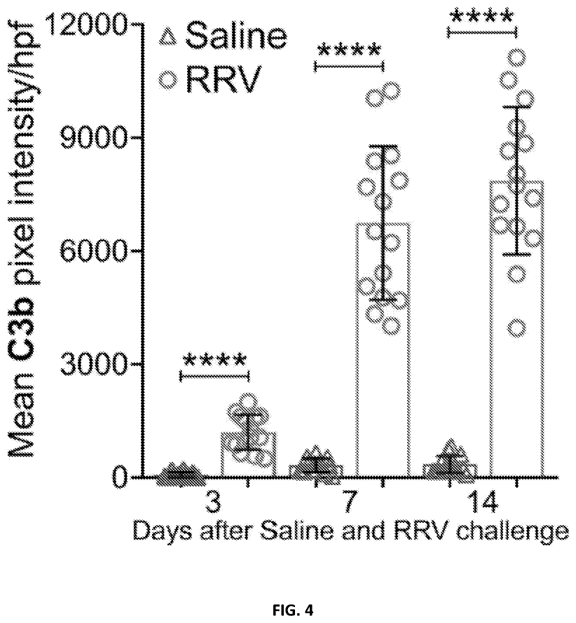

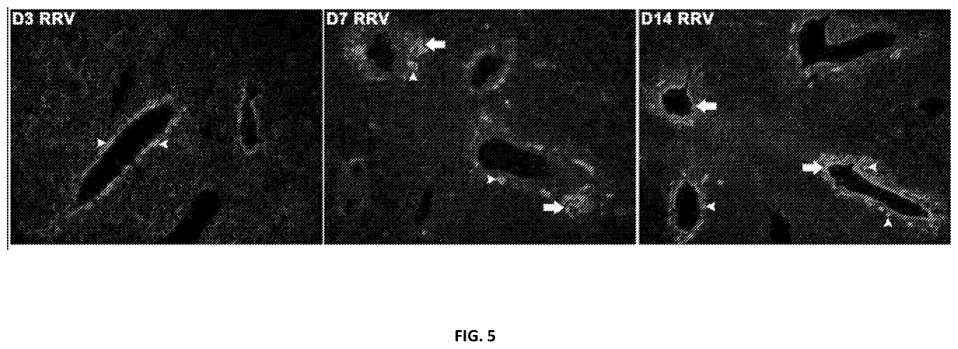

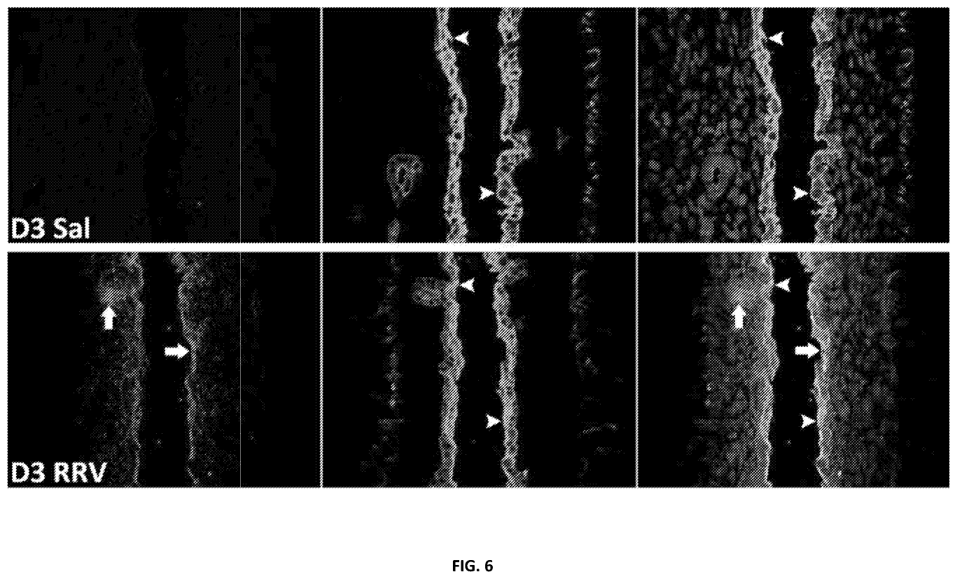

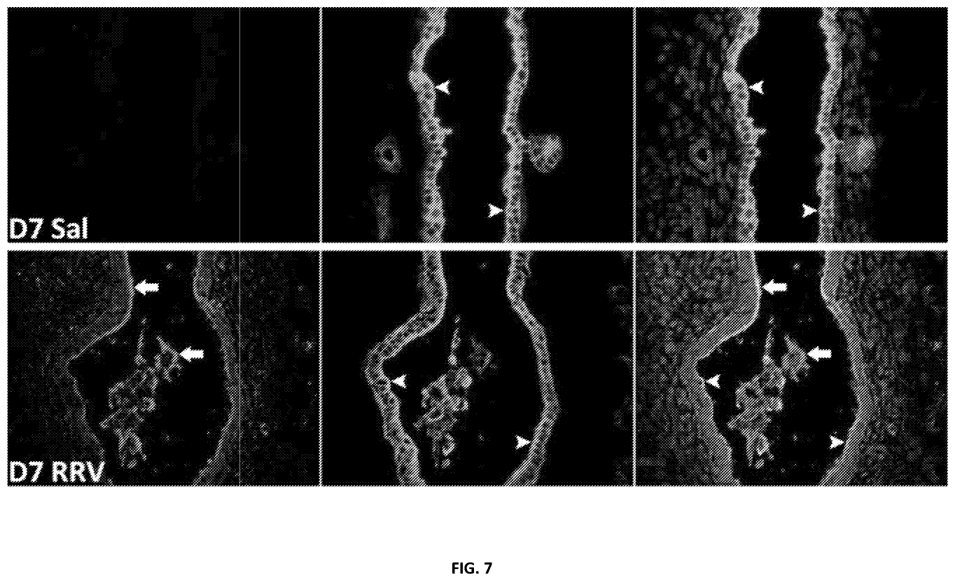

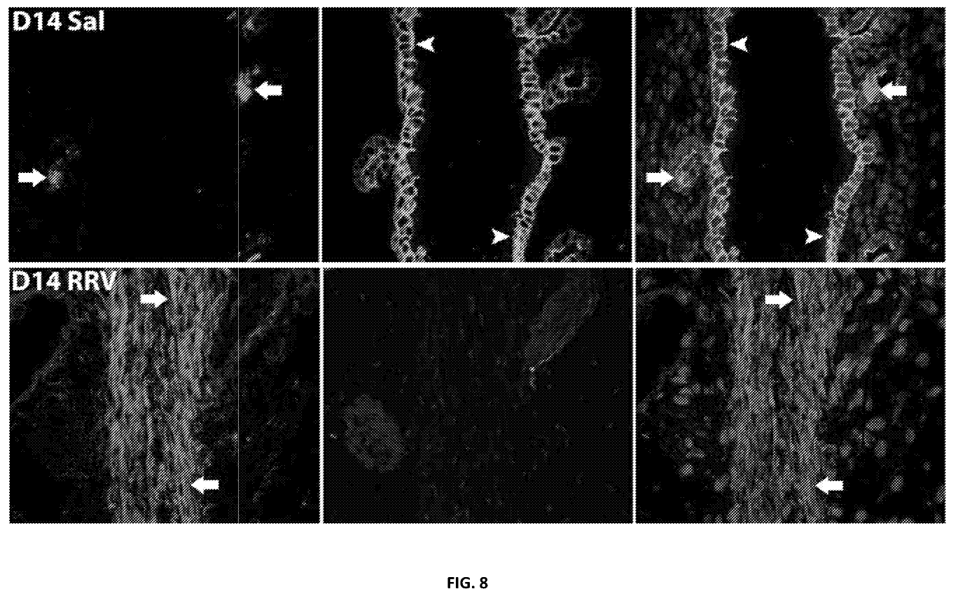

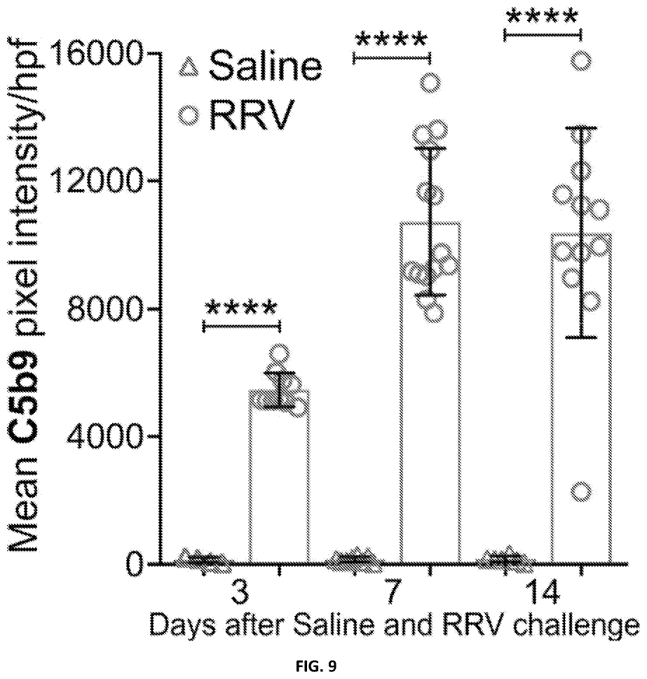

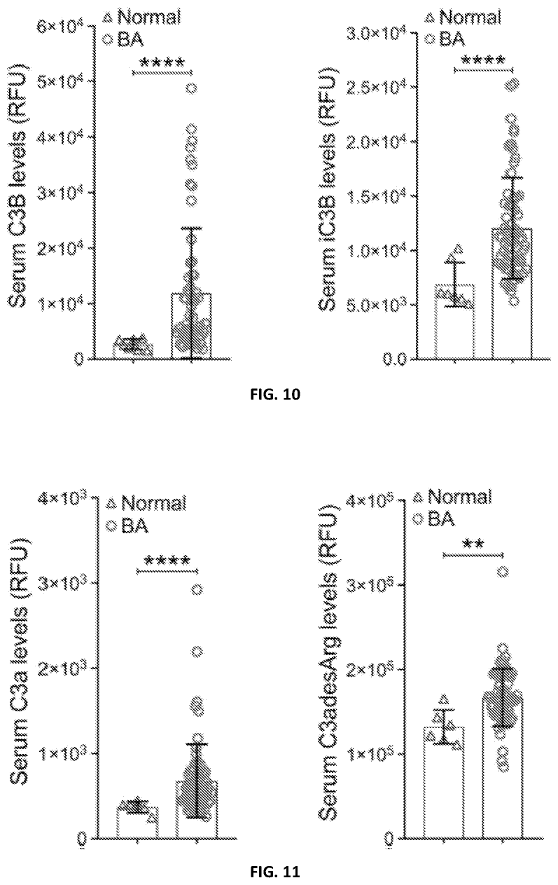

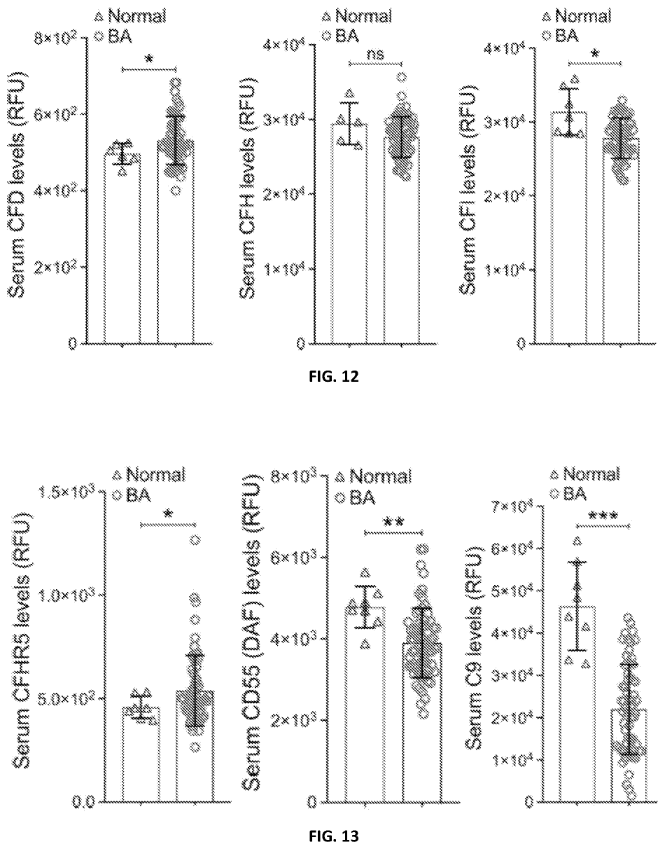

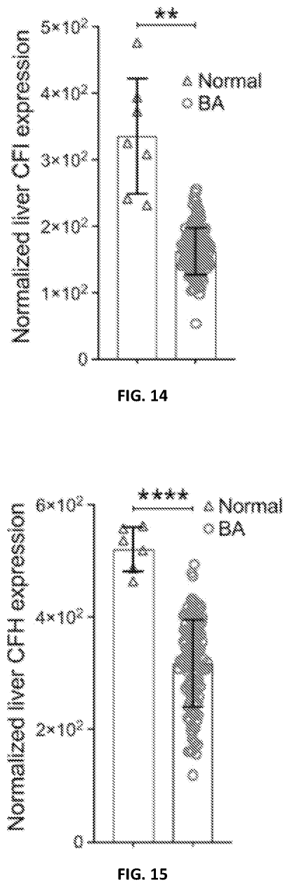

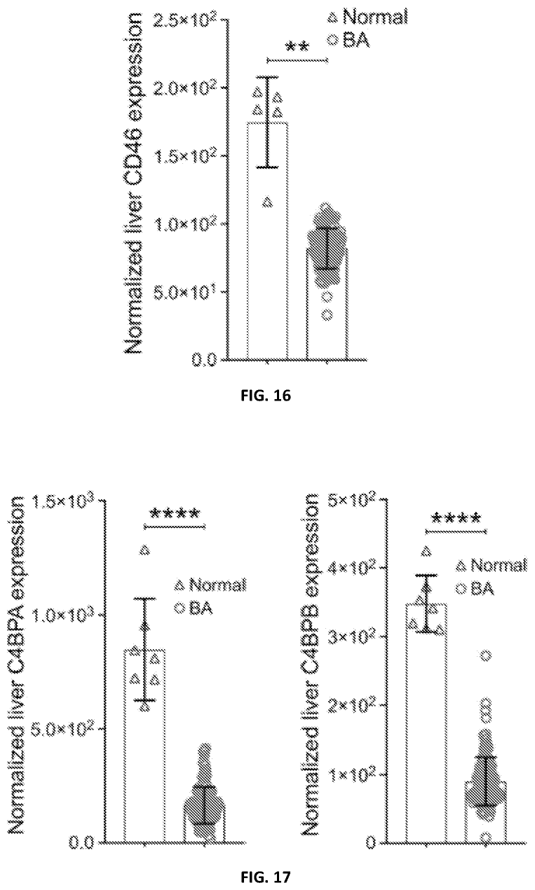

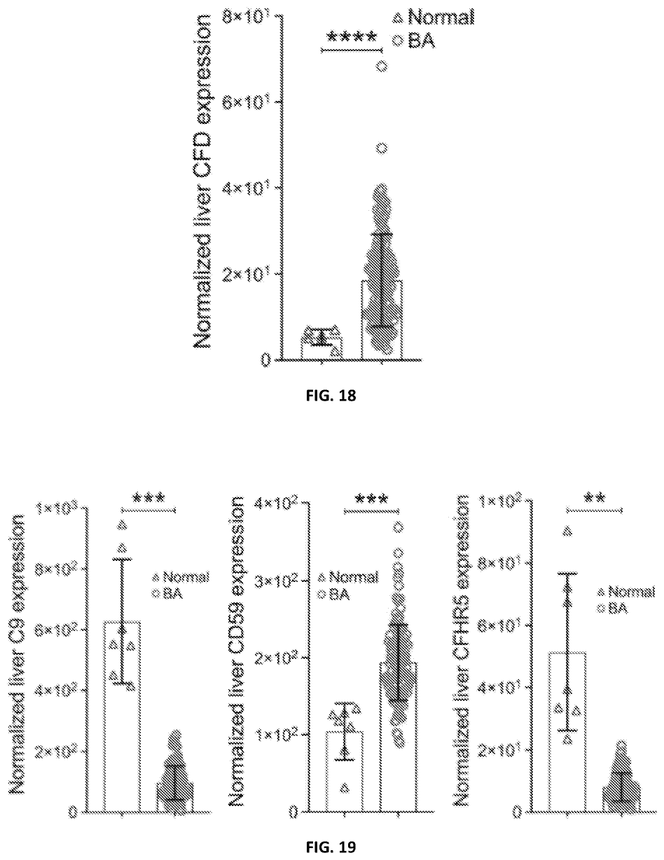

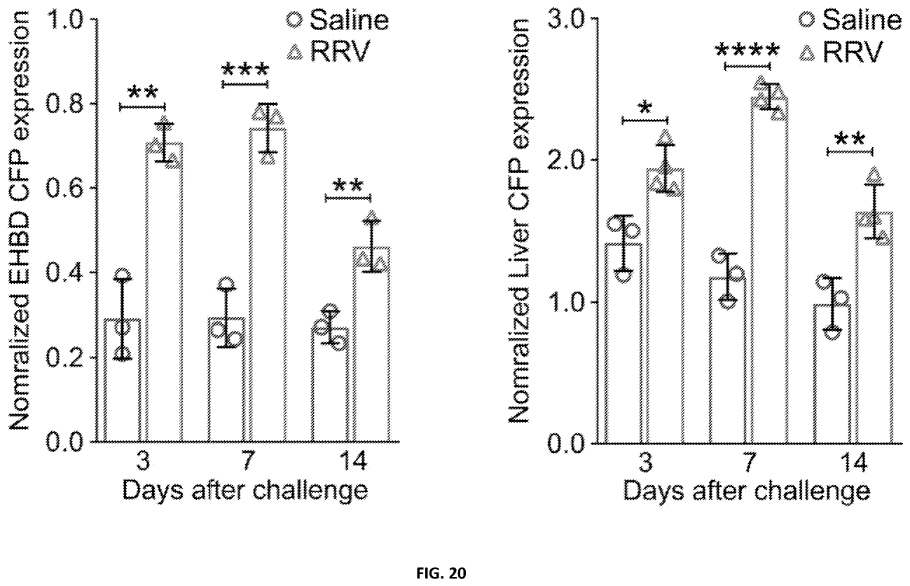

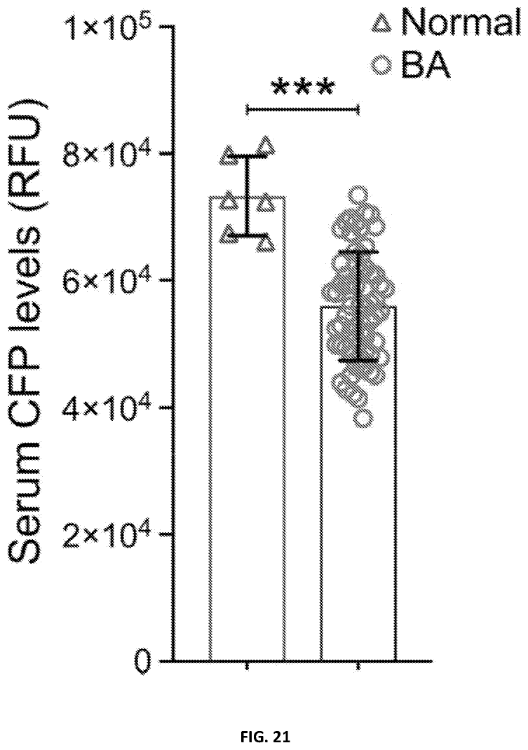

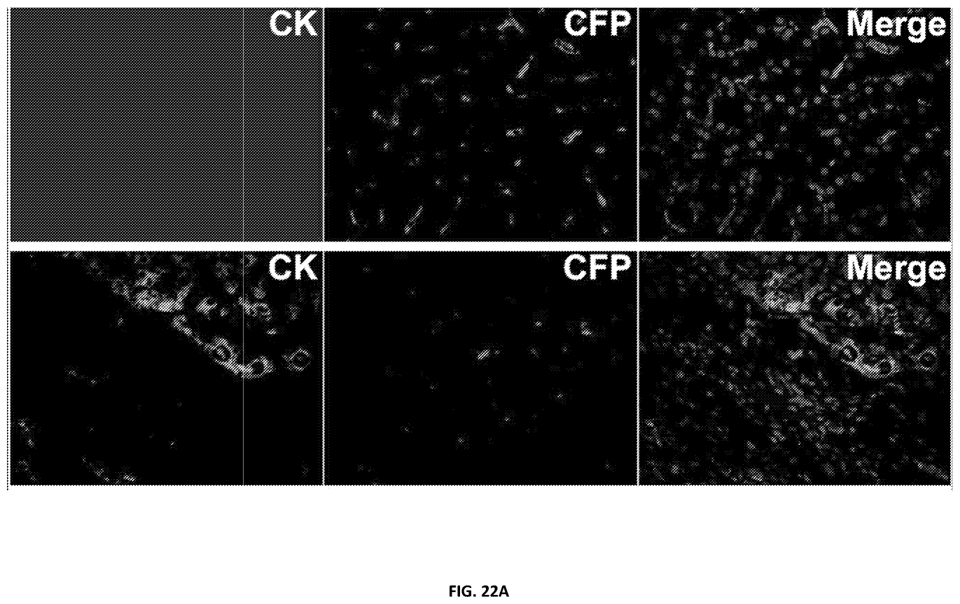

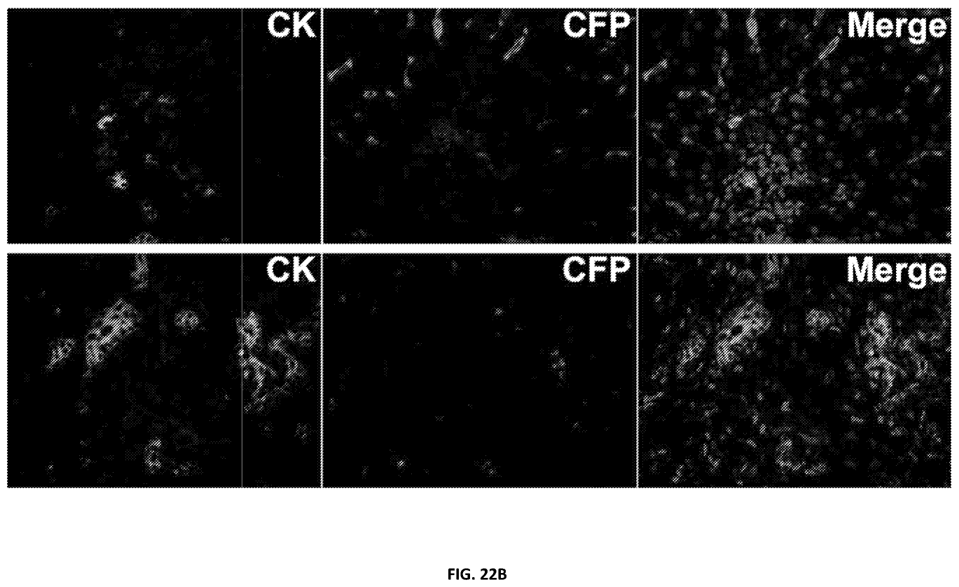

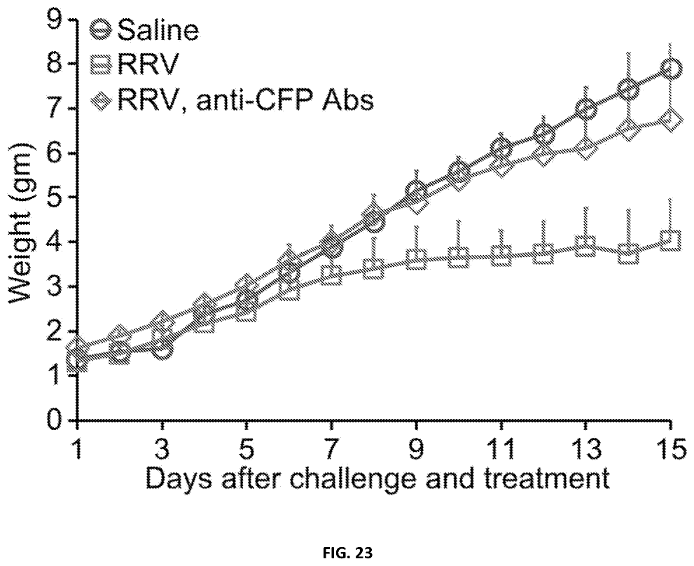

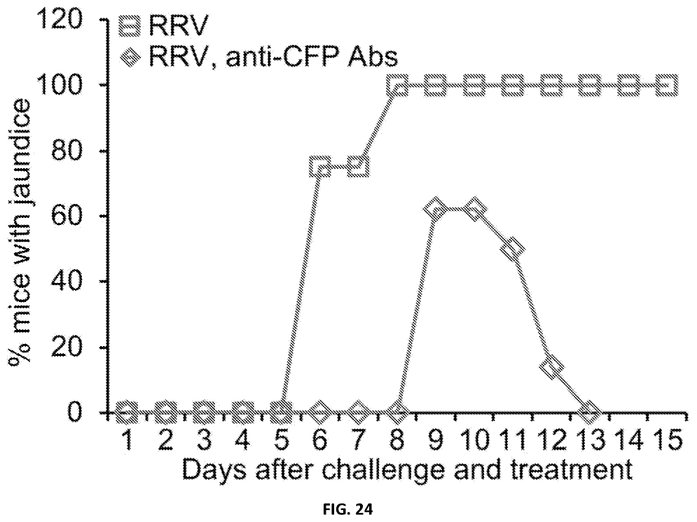

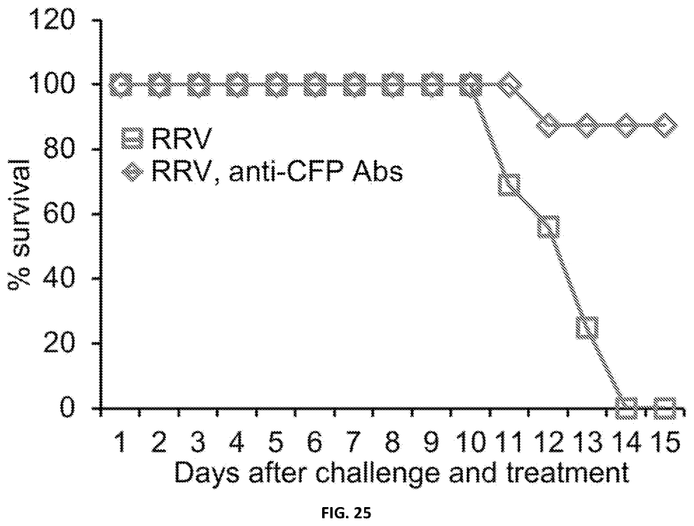

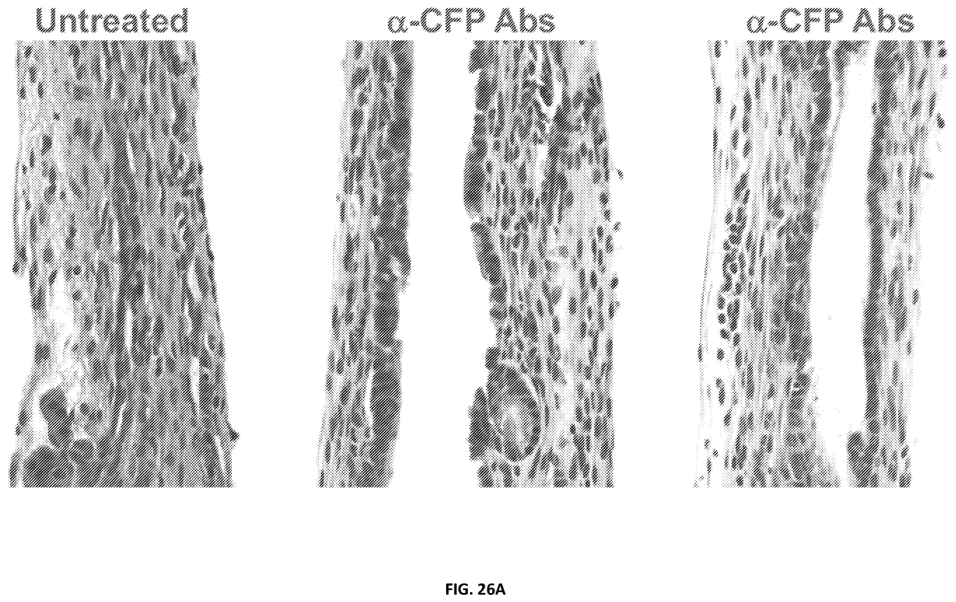

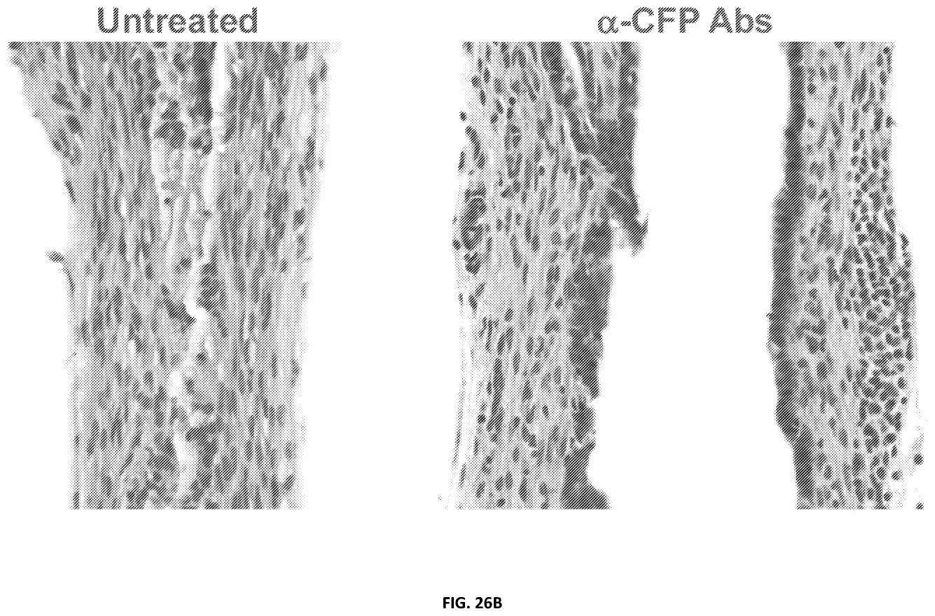

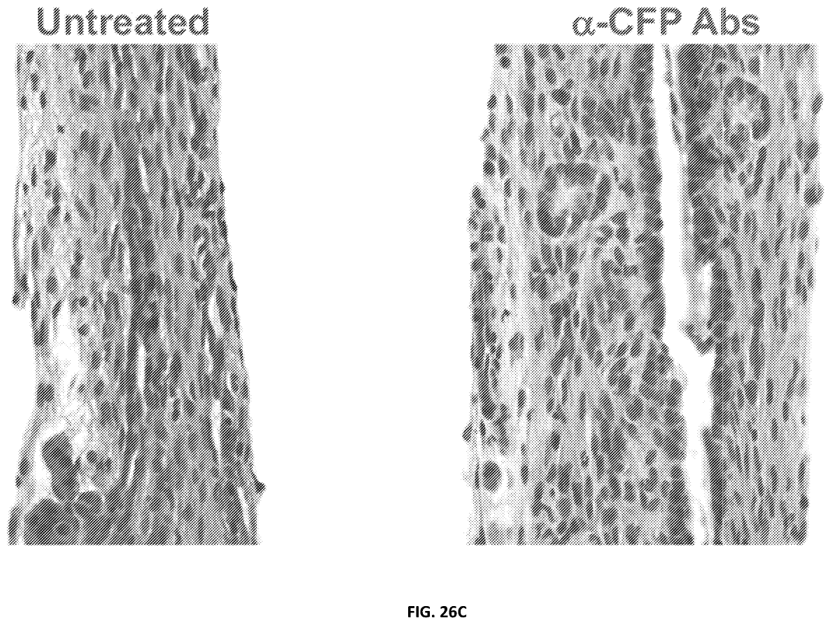

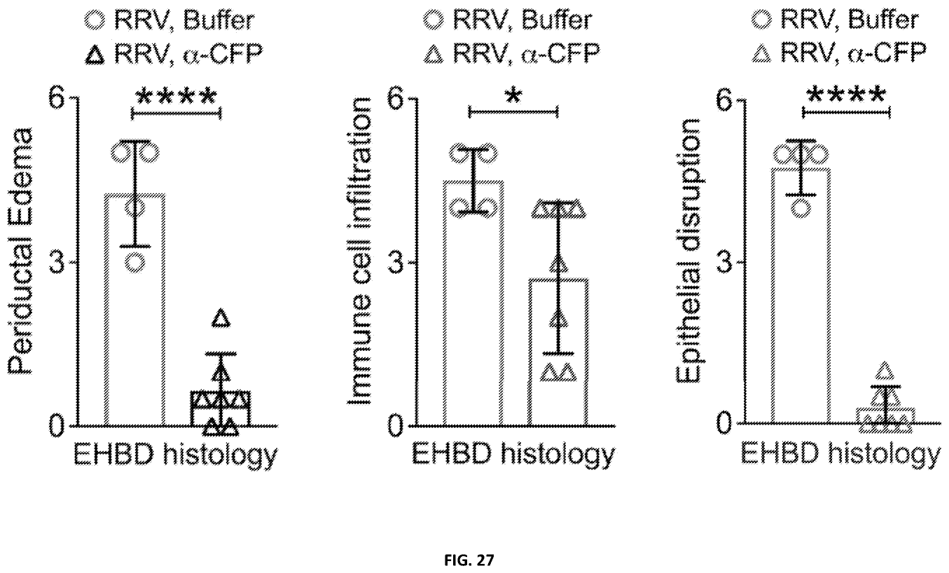

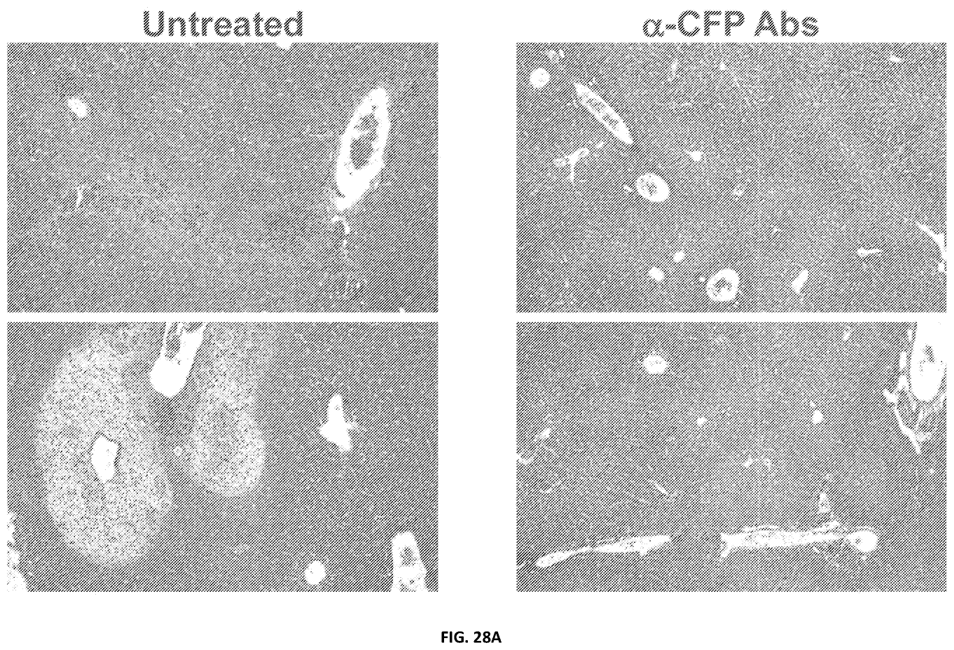

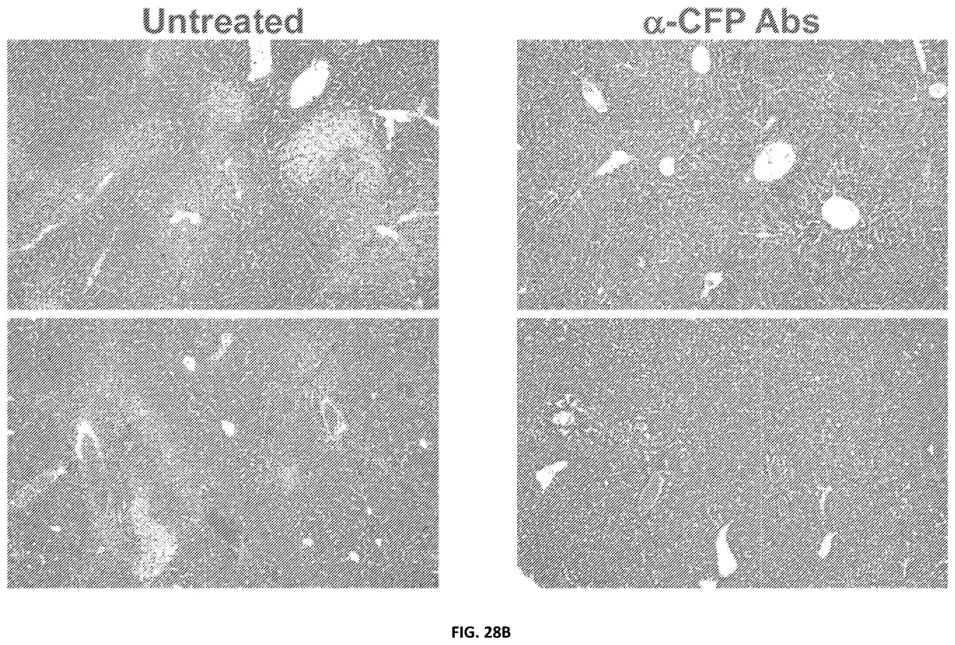

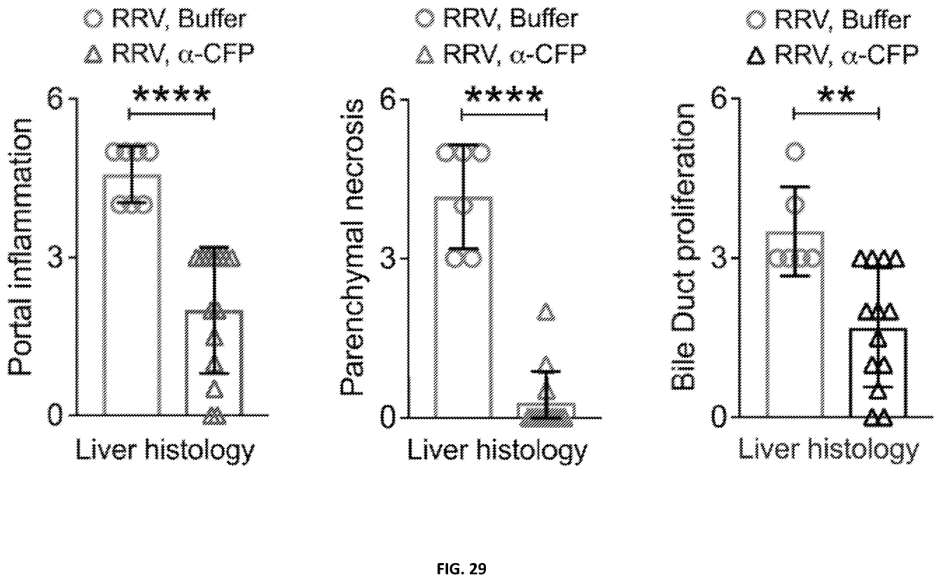

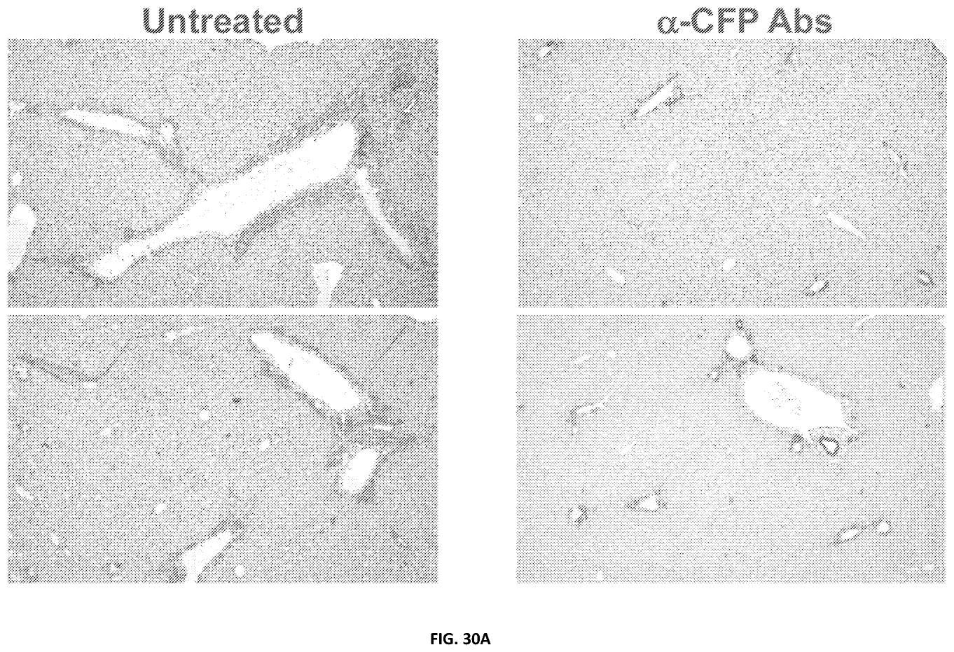

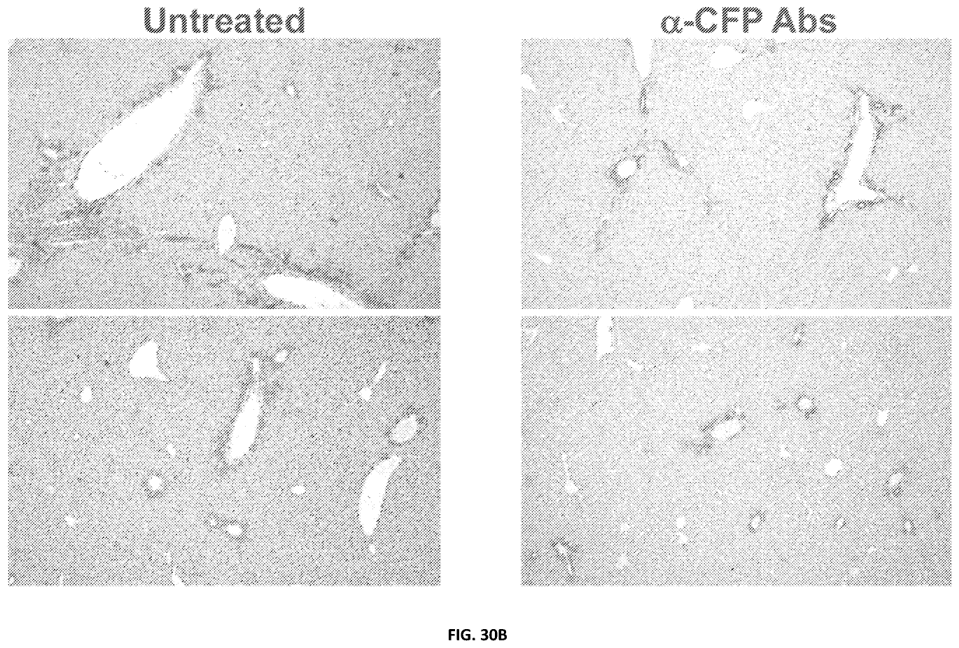

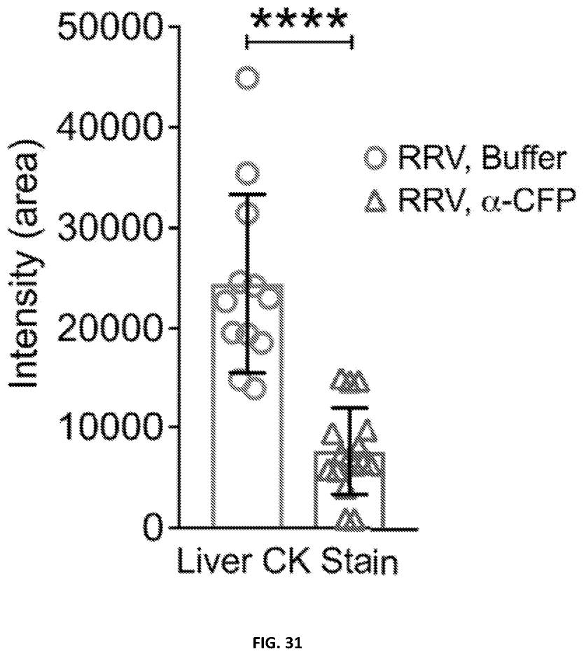

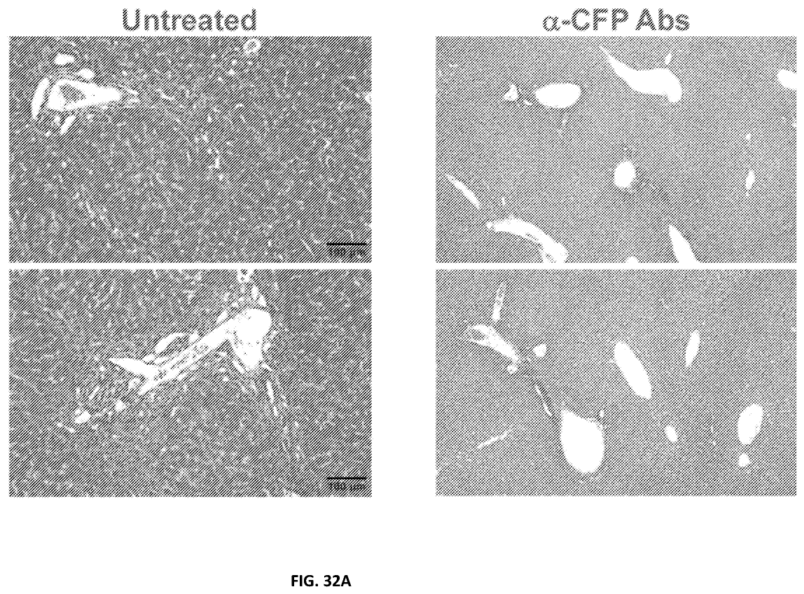

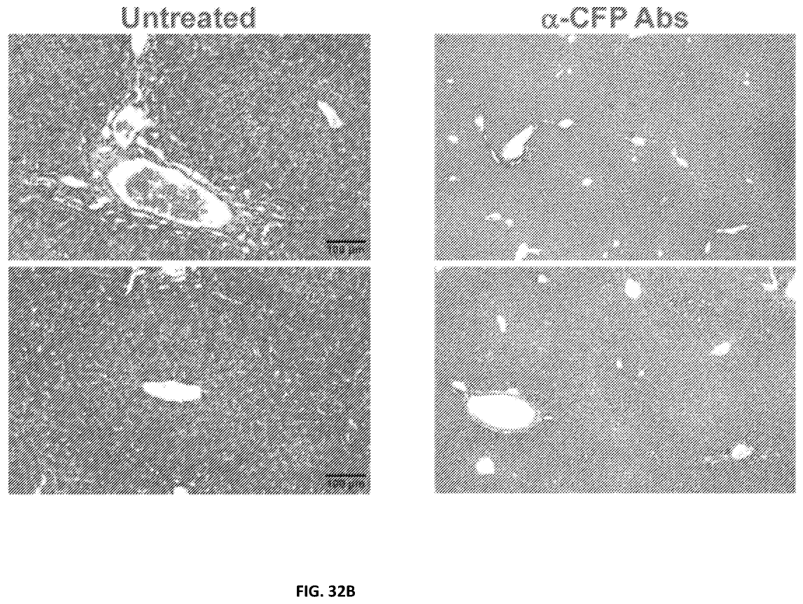

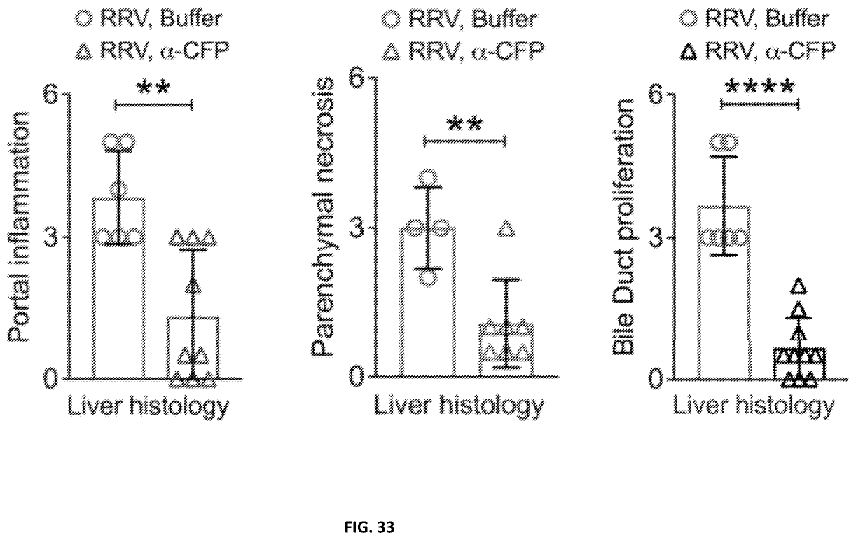

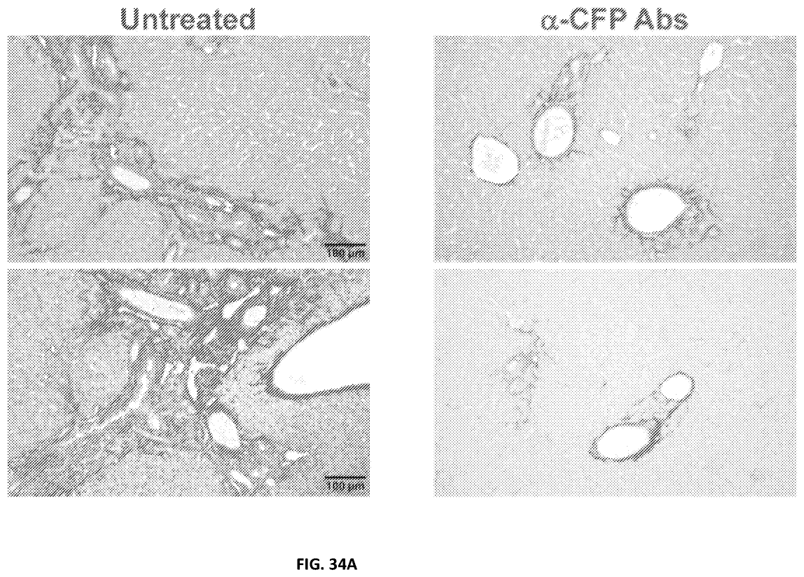

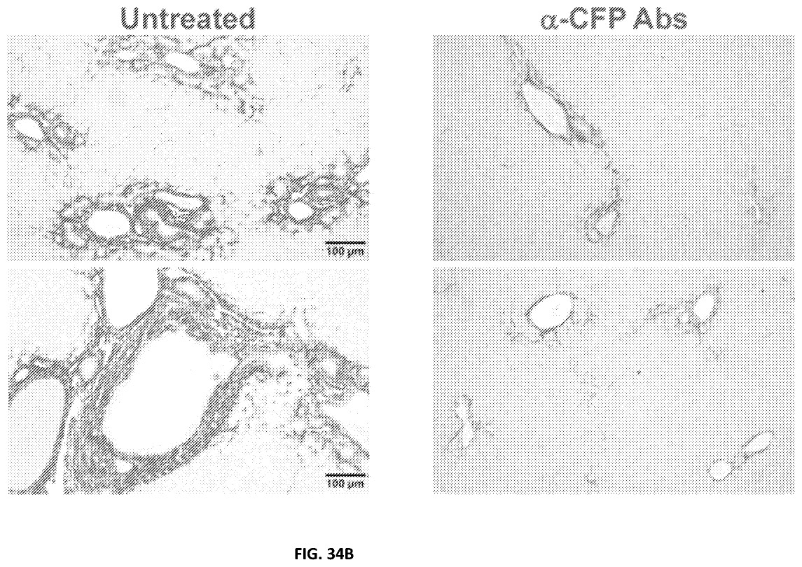

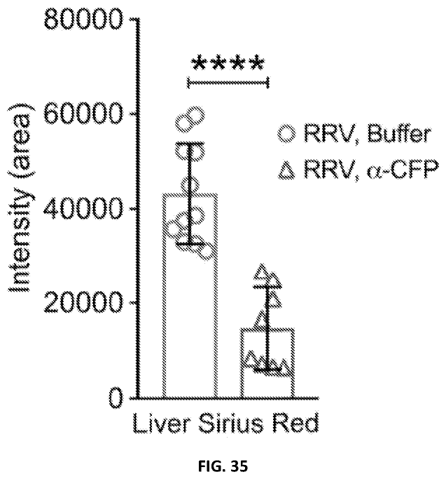

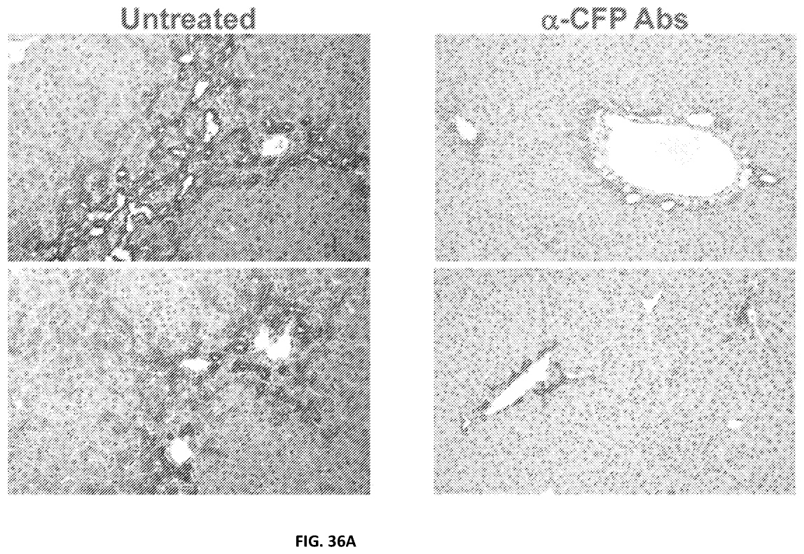

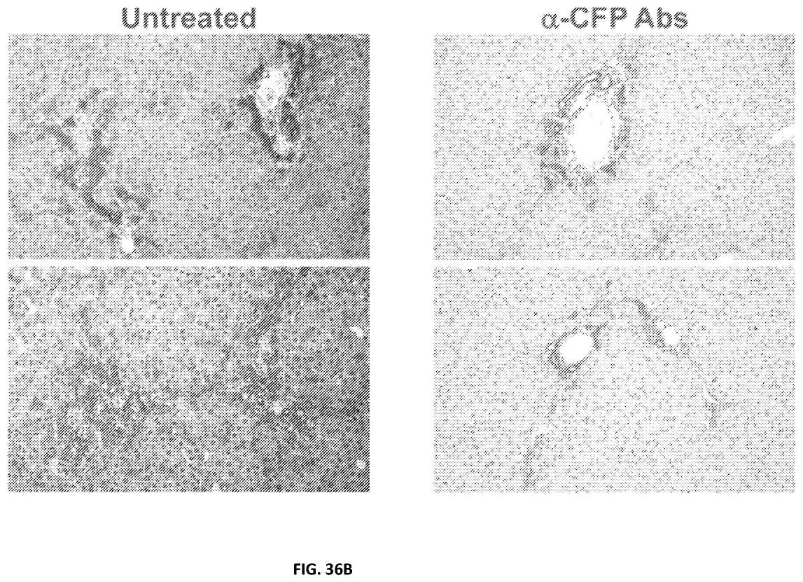

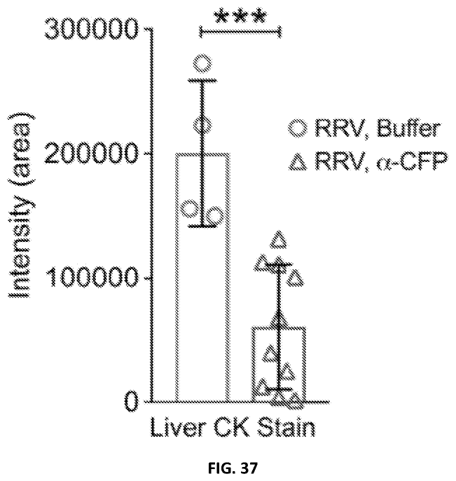

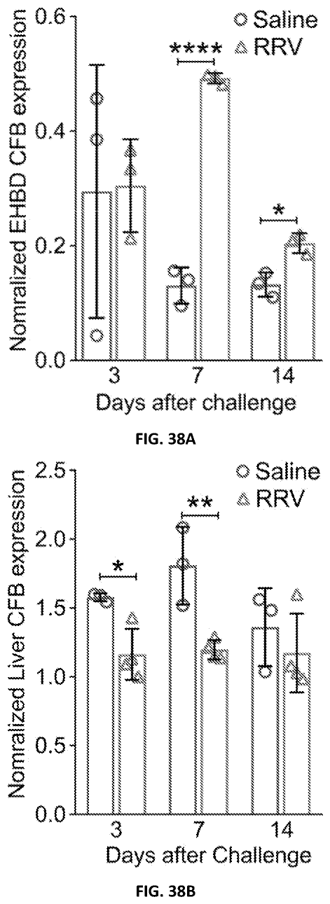

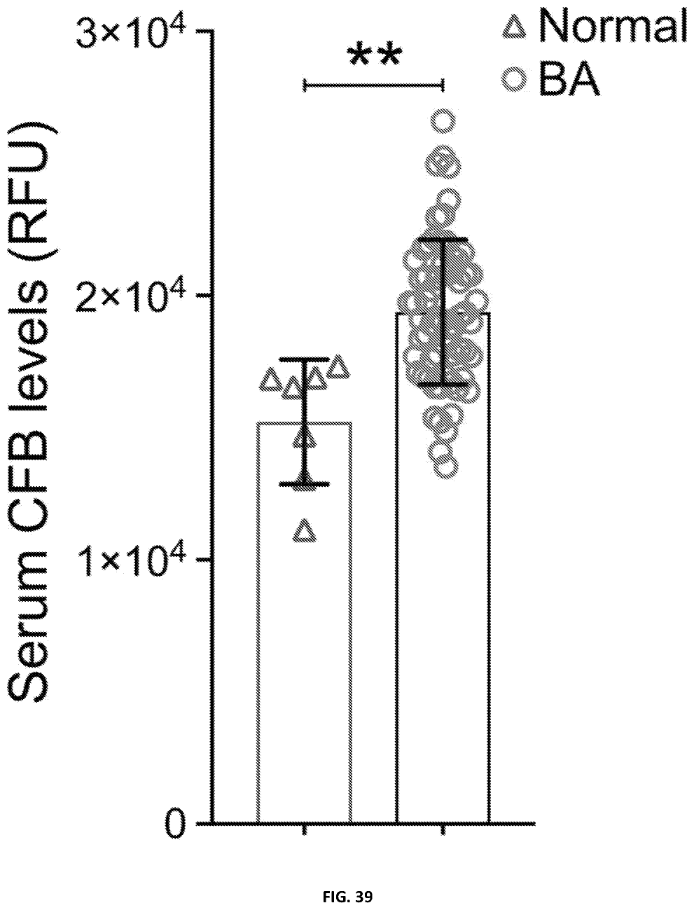

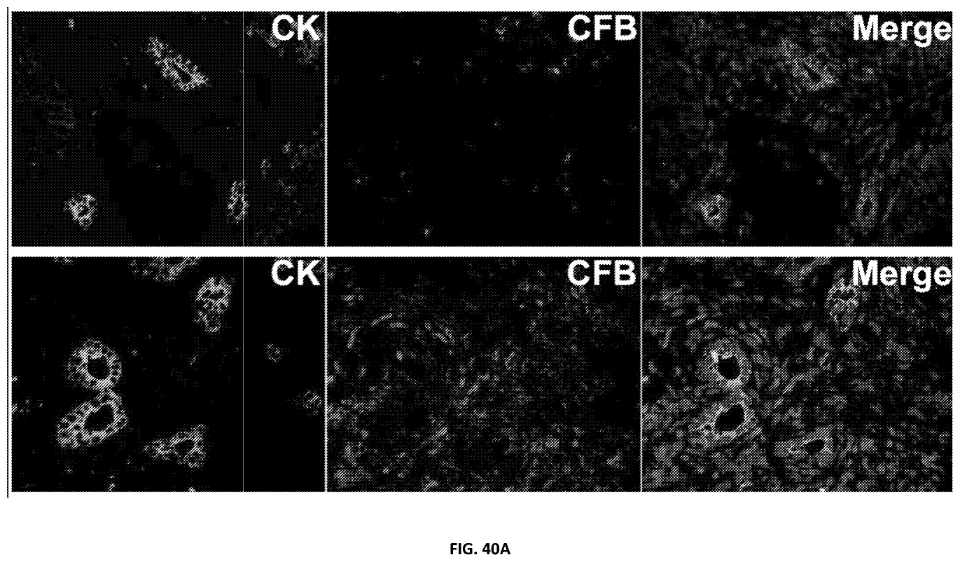

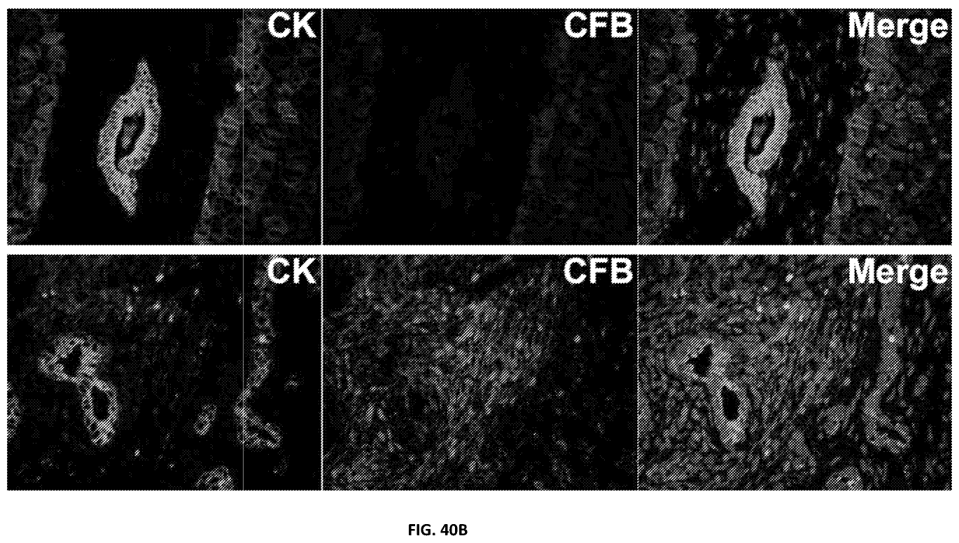

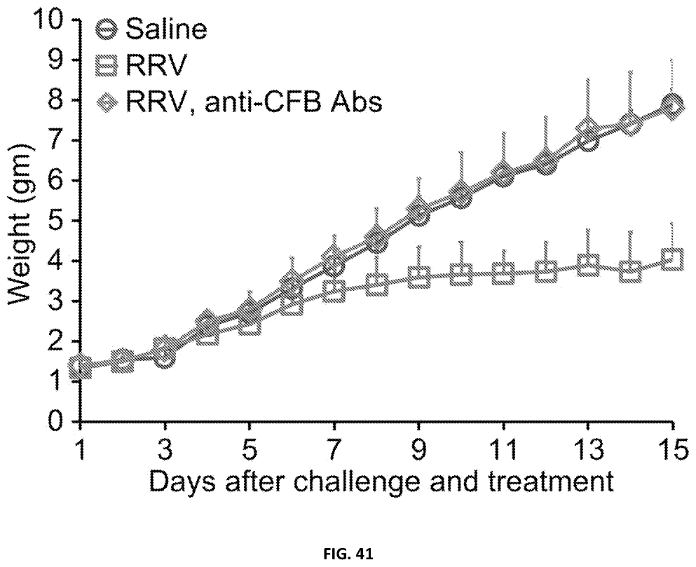

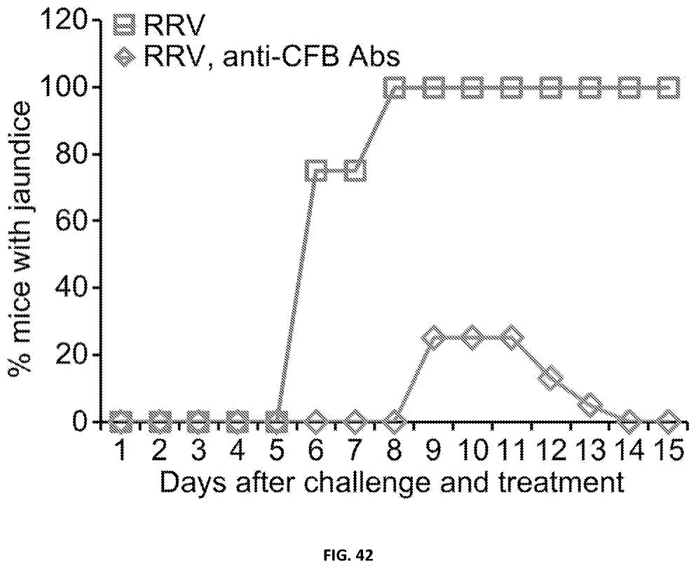

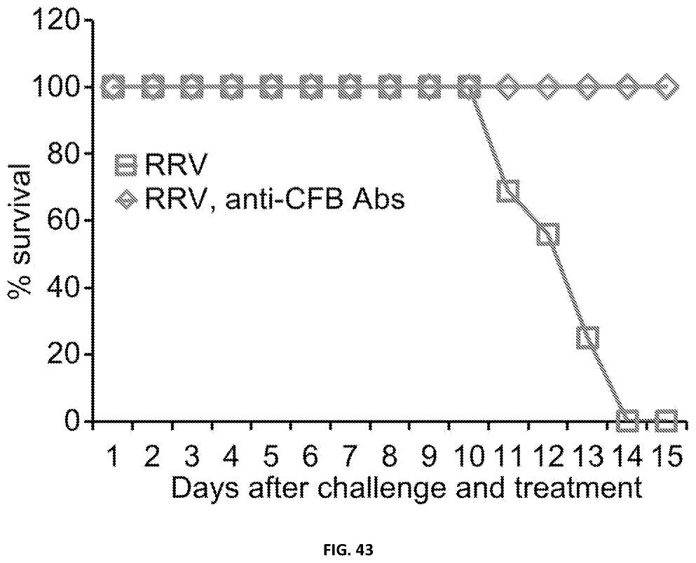

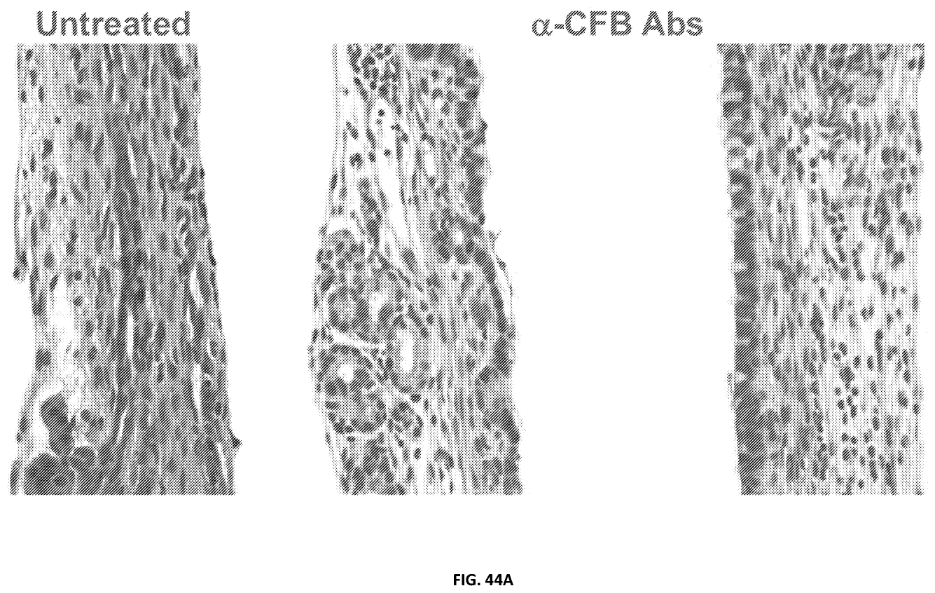

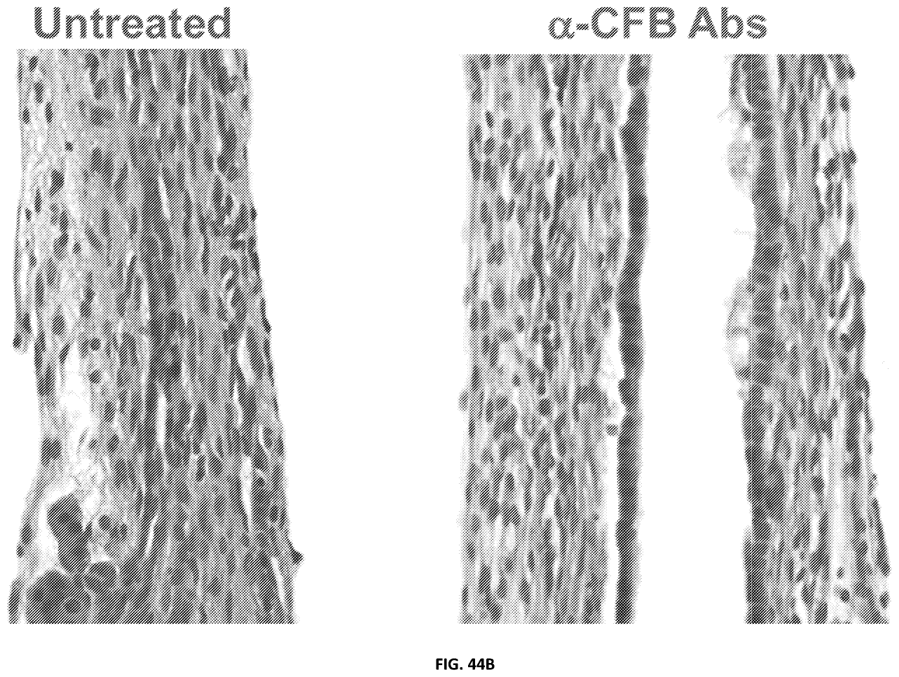

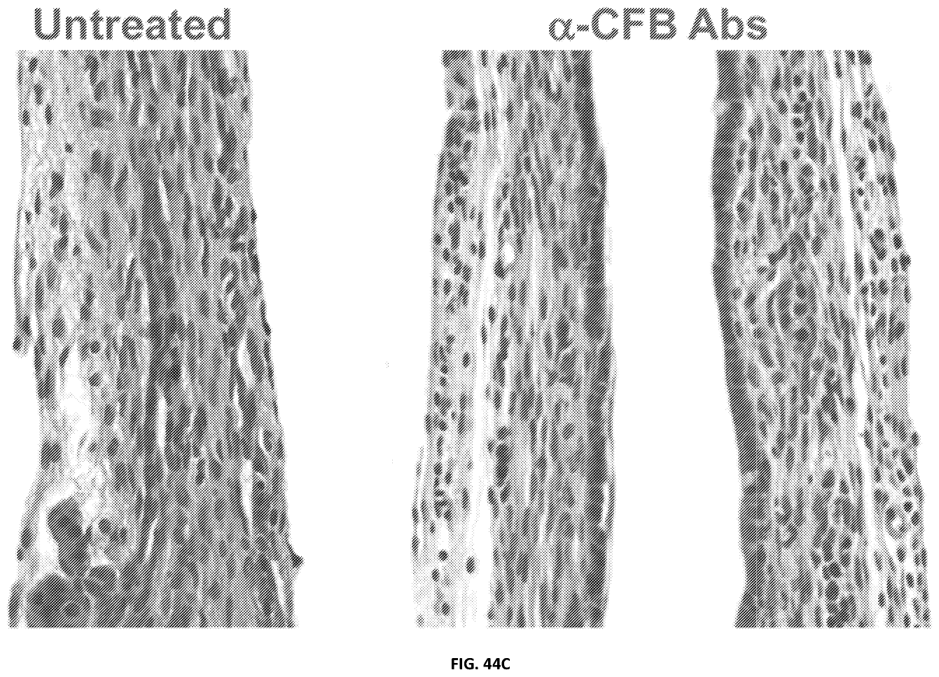

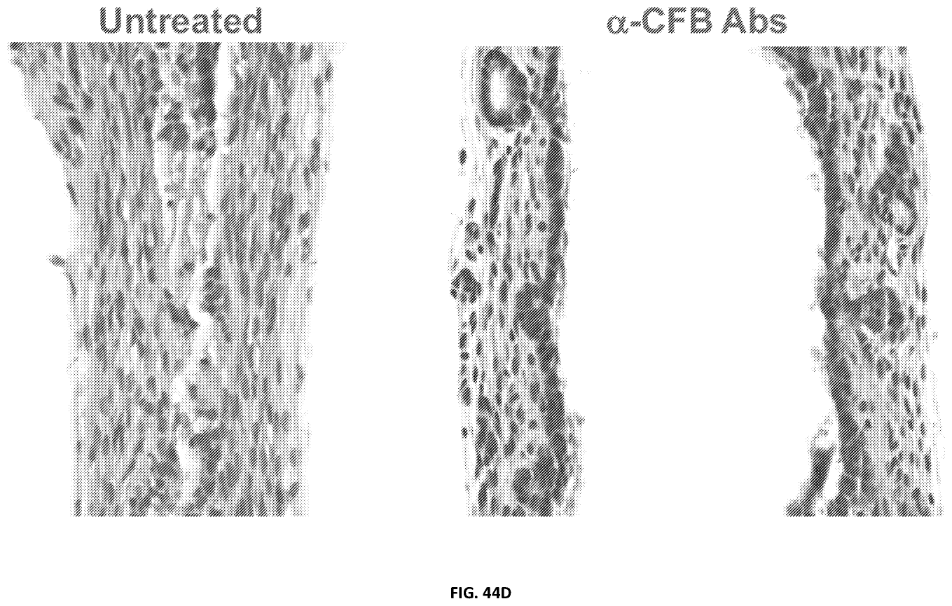

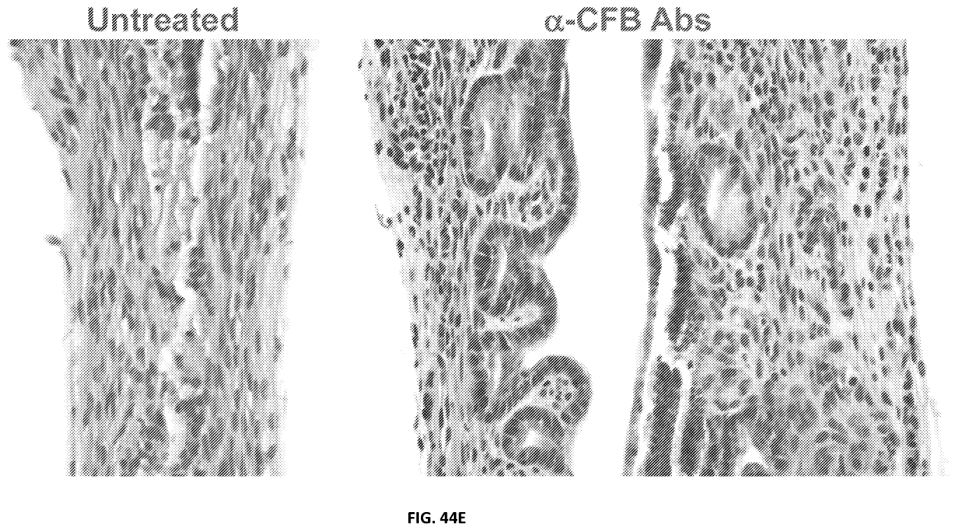

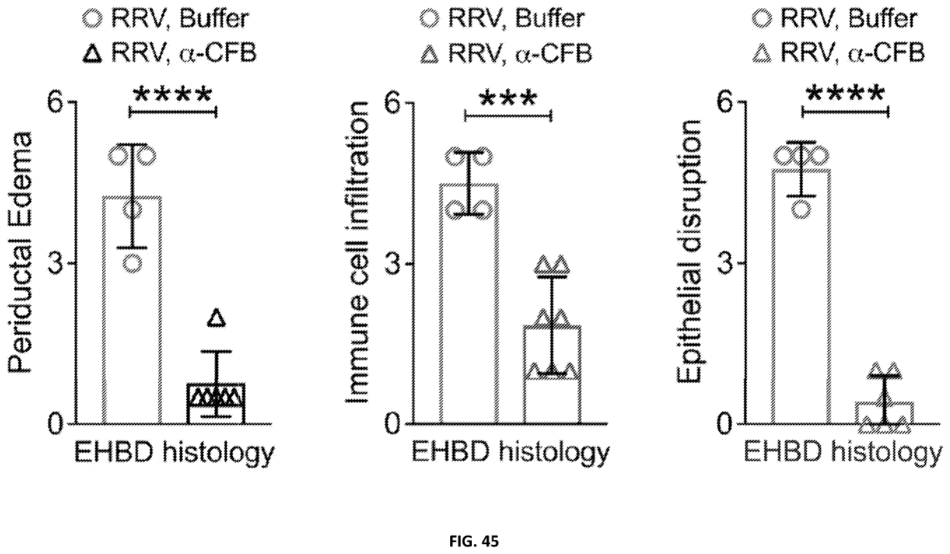

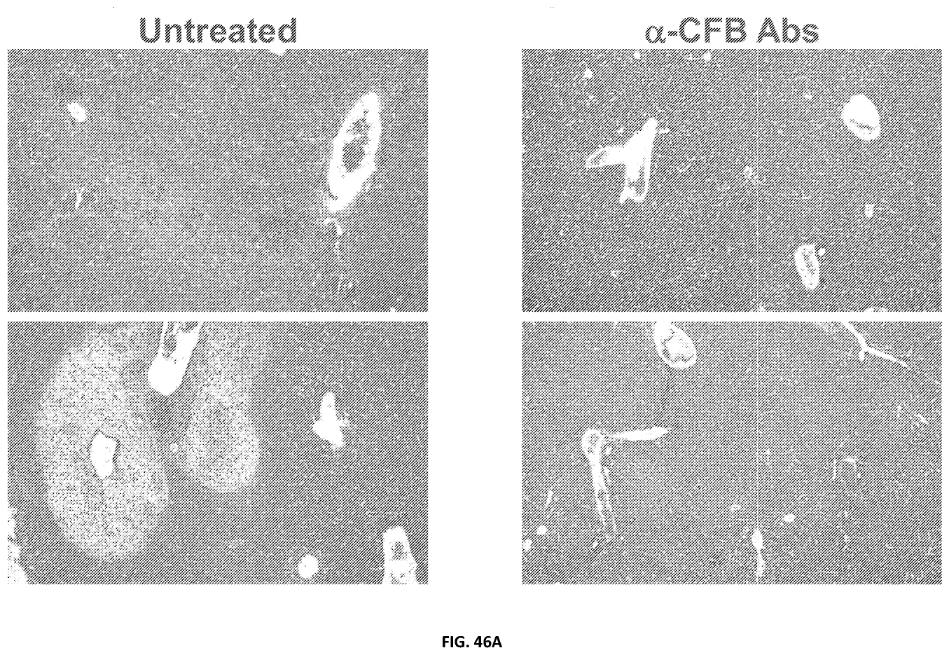

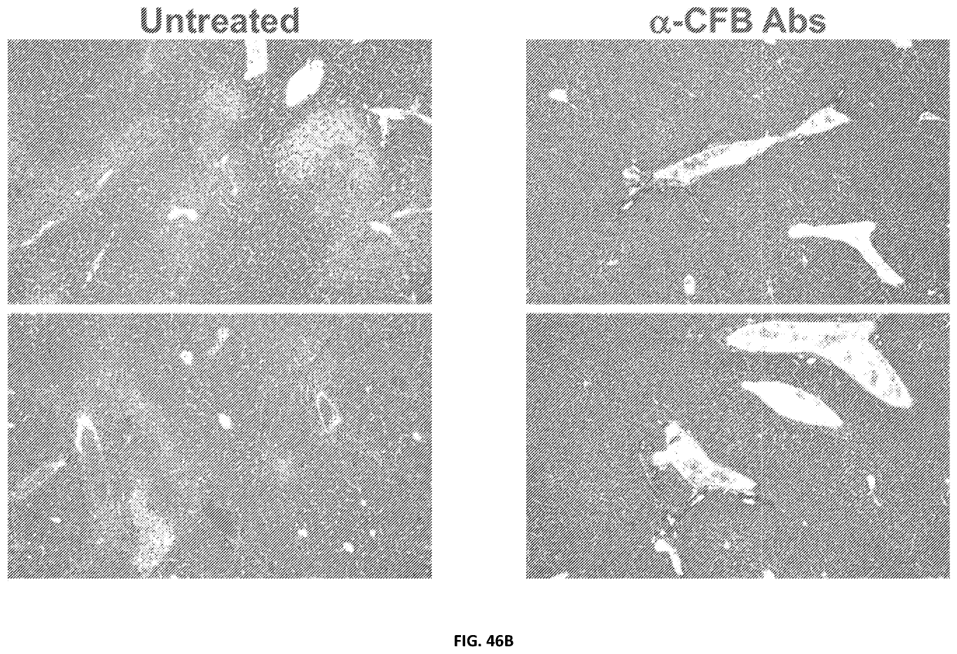

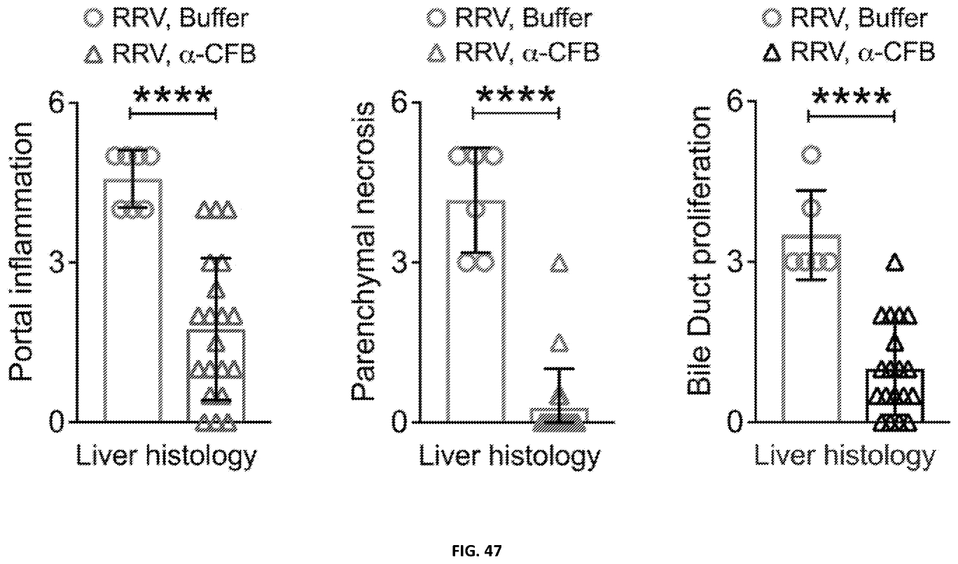

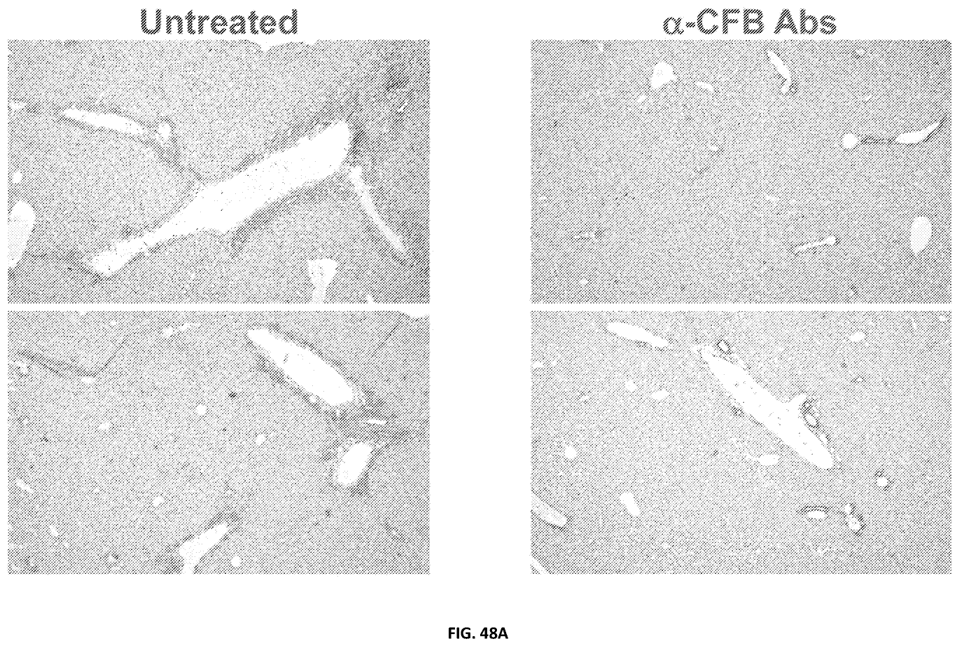

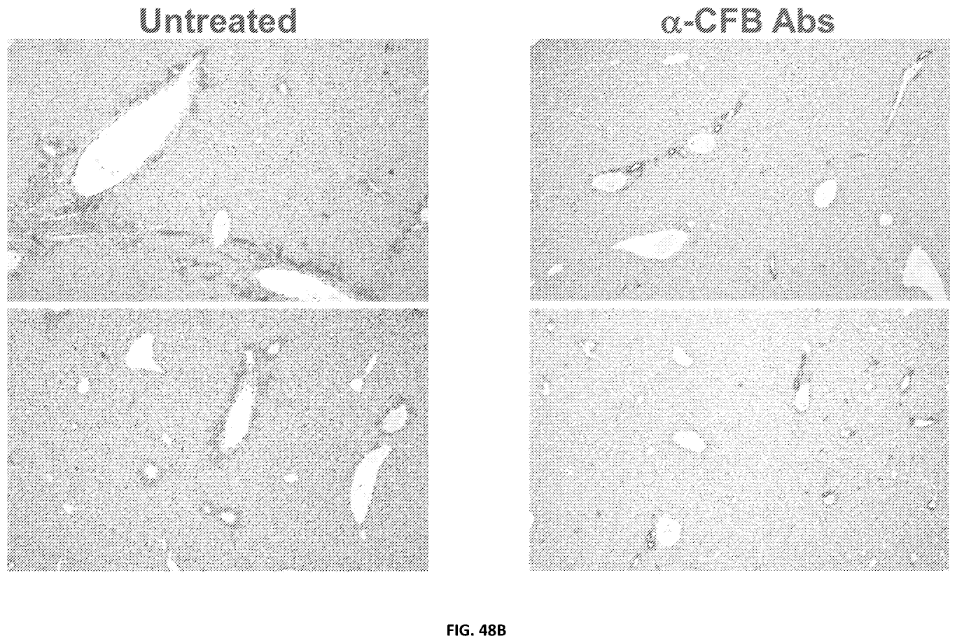

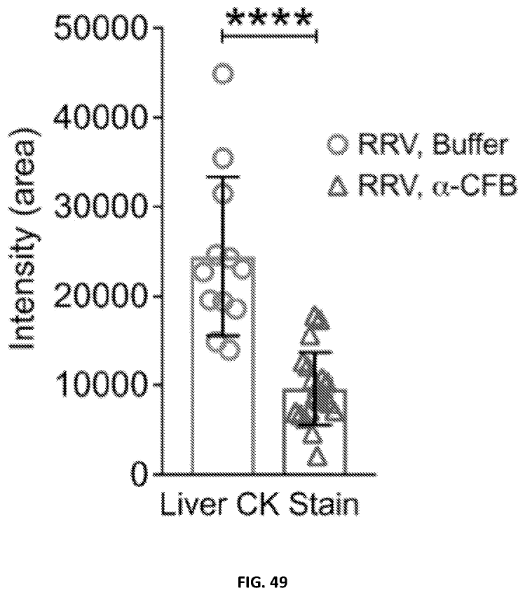

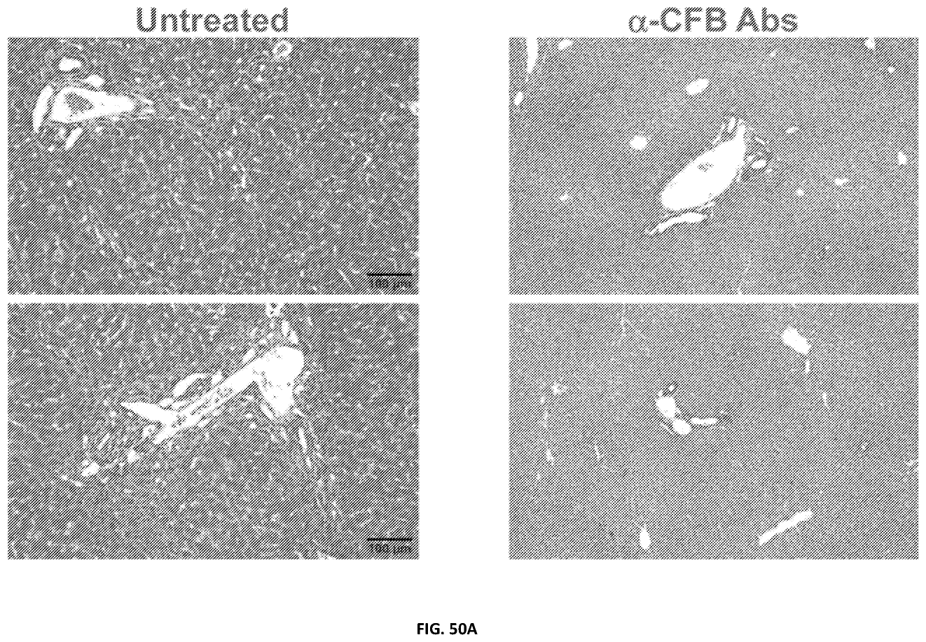

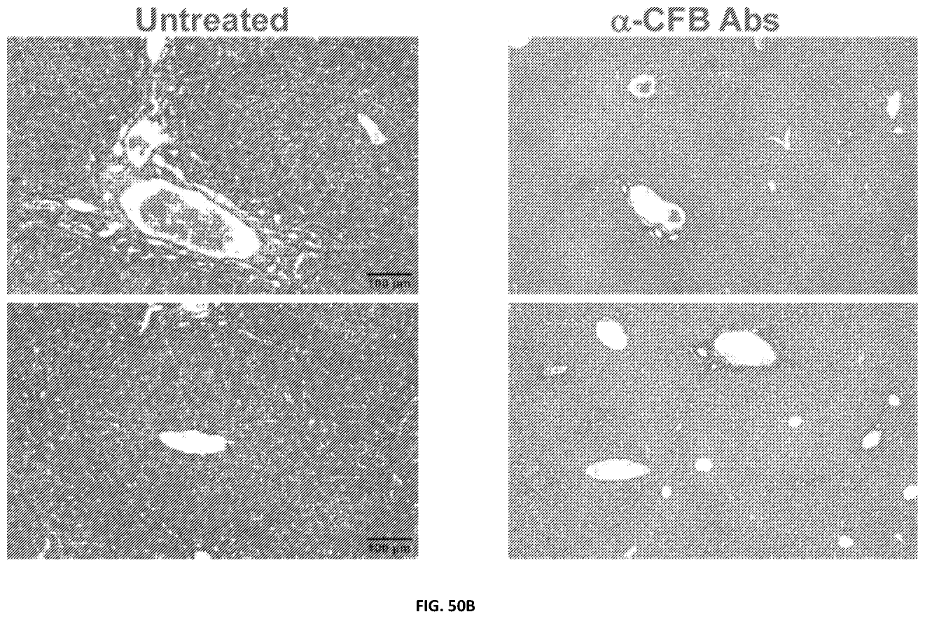

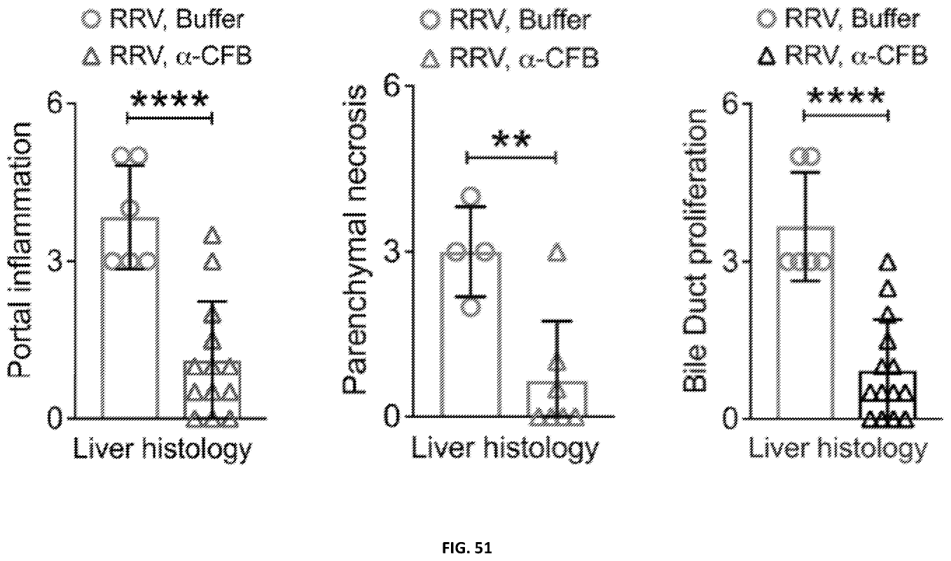

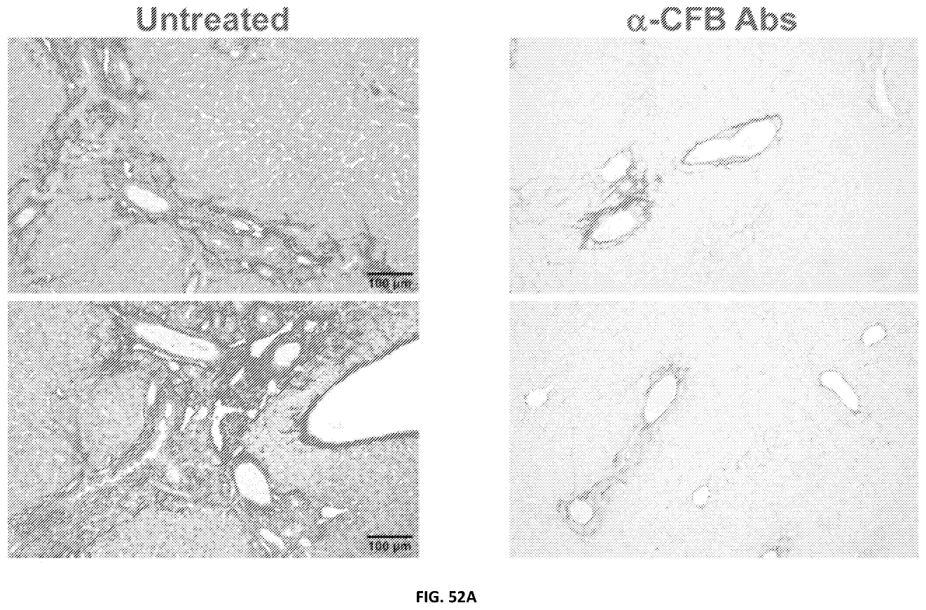

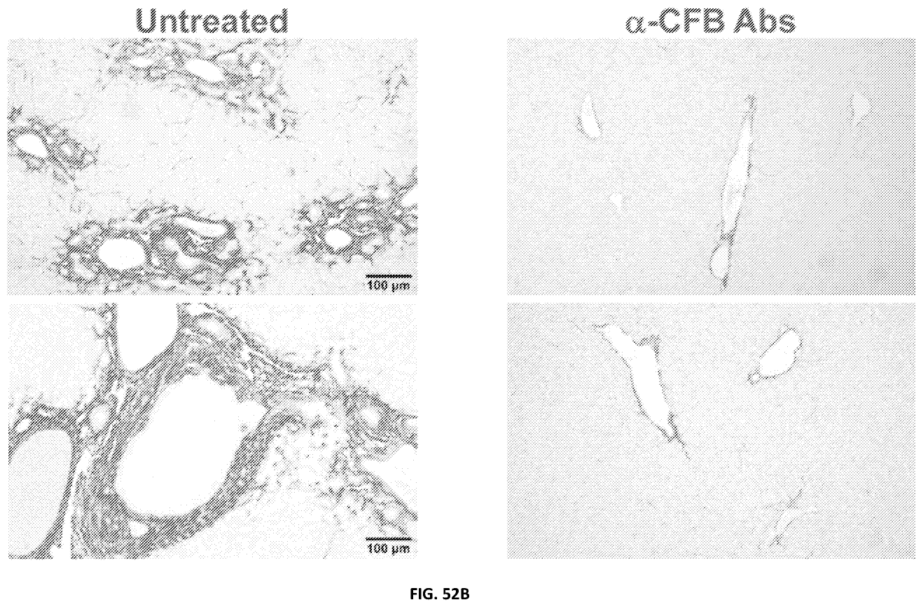

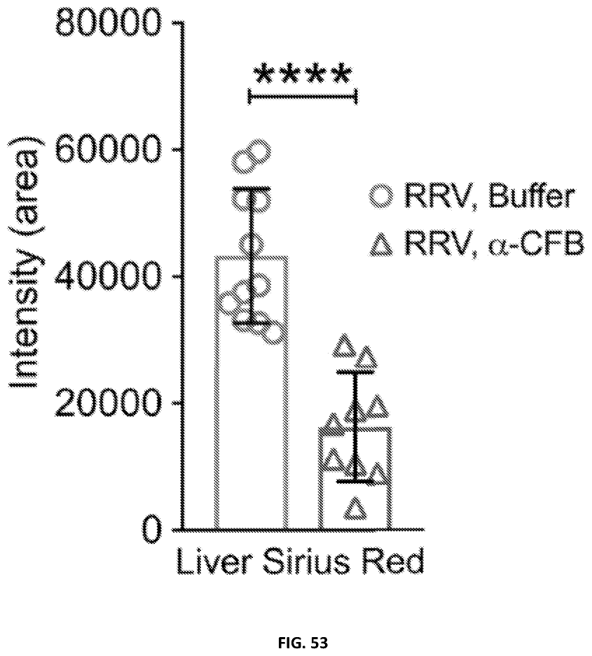

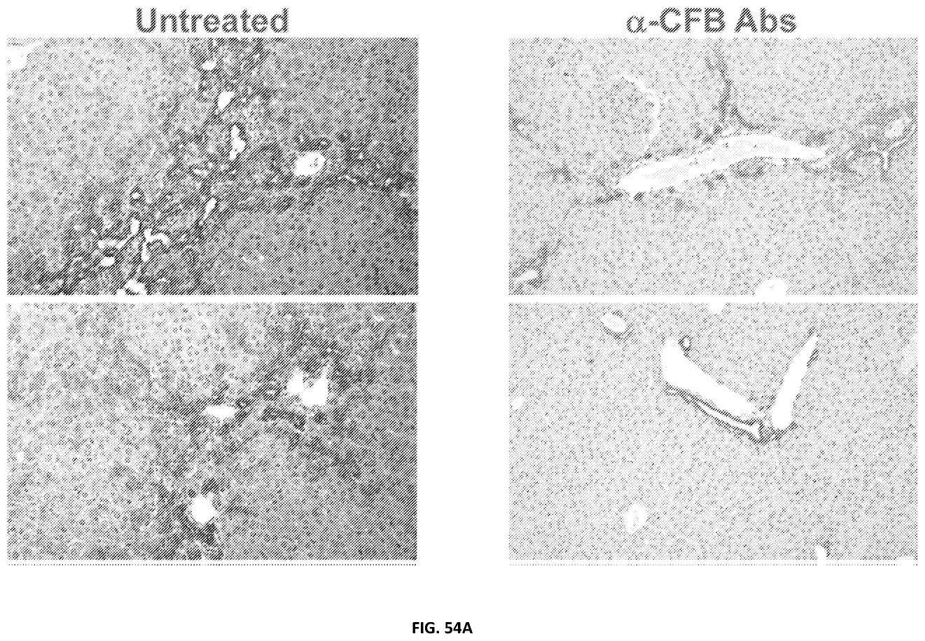

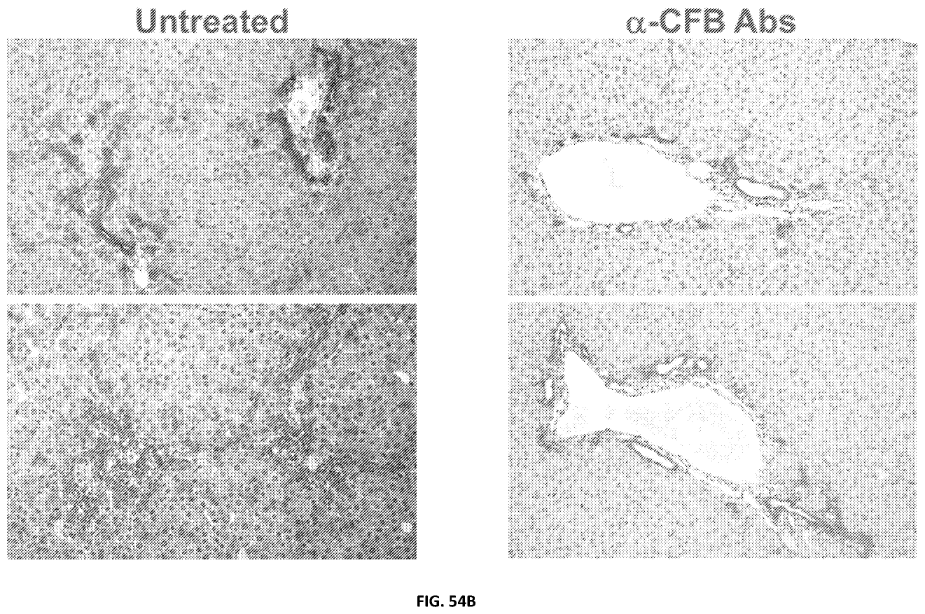

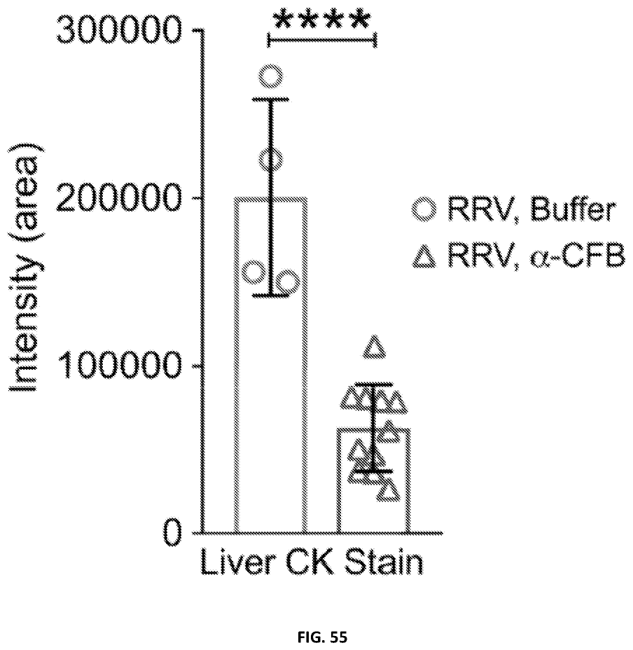

Those of skill in the art will understand that the drawings, described below, are for illustrative purposes only. The drawings are not intended to limit the scope of the present teachings in any way.

### DETAILED DESCRIPTION

Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed. As used herein, the term “antibody” refers to an immunoglobulin molecule that specifically binds to, or is immunologically reactive with, a particular antigen, and includes polyclonal, monoclonal, genetically engineered, and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen binding fragments of antibodies, including, for example, Fab′, F(ab′)₂, Fab, Fv, rlgG, and scFv fragments.

Unless otherwise indicated, the term “monoclonal antibody” (mAb) is meant to include both intact molecules, as well as antibody fragments (including, e.g., Fab and F(ab′)₂ fragments) that are capable of specifically binding to a target. As used herein, the Fab and F(ab′)₂ fragments refer to antibody fragments that lack the Fc fragment of an intact antibody. Examples of these antibody fragments are described herein.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, diabody, a triabody, an affibody, a nanobody, an aptamer, or a domain antibody. Examples of binding fragments included by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment that consists of a V_{H} domain (see, e.g., Ward et al., Nature 341 :544-546, 1989); (vii) a dAb which consists of a V_{H} or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the terms “anti-complement factor B antibody” or “anti-CFB antibody” refers to a protein or peptide-containing molecule that includes at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework region, or any portion thereof, that is capable of specifically binding to complement factor B. Anti-CFB antibodies may include CDRs, e.g., heavy chain CDRs (HCDR1-3), optionally together with light chain CDRs (LCDR1-3) that bind to CFB.

As used herein, the terms “anti-complement factor P antibody” or “anti-properdin antibody” or “anti-CFP antibody” refers to a protein or peptide-containing molecule that includes at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework region, or any portion thereof, that is capable of specifically binding to complement factor P (properdin). Anti-CFP antibodies may include CDRs, e.g., heavy chain CDRs (HCDR1-3), optionally together with light chain CDRs (LCDR1-3) that bind to CFP (properdin).

The term “preventing” is art-recognized, and when used in relation to a condition, such as a local recurrence, a disease such as biliary atresia (BA), a syndrome complex such as liver failure or any other medical condition, is well understood in the art, and includes administration of a composition which reduces the frequency of, or delays the onset of, symptoms of a medical condition in an individual relative to an individual who does not receive the composition.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). In one aspect, it is intended that the severity of the subject's condition (e.g., atresia) is reduced or at least partially improved or modified and that some alleviation, mitigation, reversal or decrease in at least one clinical symptom (e.g., weight loss in patients compared to normal subjects) is achieved.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to a pediatric individual.

Provided herein are methods for treating or ameliorating a disease characterized by or contributing to biliary fibrosis.

The disclosed methods may comprise administering an effective amount of an inhibitor of the alternative pathway of complement one or both of a Complement Factor B inhibitor and a Complement Factor P (“CFP” or “properdin”) inhibitor to an individual in need thereof. As used herein, the term “administering” means applying as a remedy, such as by the placement of a drug in a way such drug would be received, e.g., via intravenous administration, and be effective in carrying out its intended purpose. In some embodiments, the therapeutic embodiments are carried out by contacting a tissue of a subject, e.g., liver tissue, with a delivery system comprising the drug. As defined herein, “contacting” means that the composition comprising the active ingredient (e.g., anti-CFP antibody and/or anti-CFB antibody) is introduced into a sample containing a target (e.g., CFP or CFB), and incubated at a temperature and time sufficient to permit binding of the active ingredient to the target. This setup may be used in the ex vivo therapeutic context. In the in vivo therapeutic context, “contacting” means that the drug is introduced into a patient or a subject for therapy, and the drug is allowed to come in contact with the patient's target tissue, e.g., biliary tissue, in vivo.

In some embodiments, the disclosure relates to methods of treating or ameliorating a disease characterized by or contributing to biliary fibrosis, e.g., biliary atresia, comprising administering to a subject in need thereof, a composition (e.g., a pharmaceutical composition or a medicament) comprising a CFP inhibitor which is an anti-CFP antibody or an antigen-binding fragment thereof. In one aspect, the Complement Factor P (“CFP”) inhibitor may be monoclonal antibody 14E1 (mAb14E1) or an antigen-binding fragment thereof, as described in Miwa et al. (J Immunol. 2013 Apr. 1; 190(7):3552-9; PMID: 23427256); Ueda et al. (J Am Soc Nephrol. 2018 July; 29(7):1928-1937; PMID: 29858280), the entirety of the disclosures in these publications are incorporated by reference herein.

In some embodiments, the disclosure relates to methods of treating or ameliorating a disease characterized by or contributing to biliary fibrosis, e.g., biliary atresia, comprising administering to a subject in need thereof, a composition (e.g., a pharmaceutical composition or a medicament) comprising a CFB inhibitor which is an anti-CFB antibody or an antigen-binding fragment thereof. In one aspect, the Complement Factor B (“CFB”) inhibitor may be the inhibitory monoclonal anti-factor B antibody 1379 (“mAb 1379”) or an antigen-binding fragment thereof, as described in Thurman et al. (Mol Immunol. 2005 January; 42(1):87-97; PMID: 15488947); Thurman et al. (J Am Soc Nephrol. 2006 March; 17(3):707-15; PMID: 16467447); and Leinhase et al. (J Neuroinflammation. 2007 May 2; 4:13; PMID: 17474994), the entirety of the disclosures in these publications are incorporated by reference herein. In a further aspect, the CFB inhibitor may be a humanized anti-CFB antibody or fragment or derivative thereof as described herein. “Humanized” antibodies is an art-recognized term and refers to an antibody comprising heavy and light chain variable region sequences from a non-human species (e.g., a mouse) but in which at least a portion of the VH and/or VL sequence has been altered to be more “human-like”, i.e., more similar to human germline variable sequences.

In one aspect, the disclosure relates to methods of treating or ameliorating a disease characterized by or contributing to biliary fibrosis, e.g., biliary atresia, comprising administering to a subject in need thereof, a composition (e.g., a pharmaceutical composition or a medicament) comprising the anti-CFB antibody (e.g., mAb 1379) or an antigen-binding fragment thereof and the anti-CFP antibody (e.g., mAb 14E1) or an antigen-binding fragment thereof. Pharmaceutical compositions of the disclosure typically comprise one or more of the aforementioned inhibitors, e.g., anti-CFB antibody or anti-CFB antibody or the respective antigen-binding fragment(s) thereof, and a pharmaceutically acceptable carrier. As used herein, the term “pharmaceutically acceptable” is understood to mean not biologically or otherwise undesirable, i.e., the material can be administered to a subject without causing any undesirable biological effects such as toxicity. In some embodiments, the formulations of the disclosure can optionally comprise pharmaceutical agents, carriers, adjuvants, dispersing agents, diluents, and the like. The pharmaceutical compositions can be formulated for administration in accordance with known techniques. See, e.g., Remington, The Science & Practice of Pharmacy (9th Ed., 1995). In the manufacture of a pharmaceutical compositions, the inhibitor is typically admixed with, inter alia, an acceptable carrier. The carrier can be a solid or a liquid and may be formulated with the inhibitor as a unit-dose formulation.

The antibody may be specific to the component and may inhibit the component's function or by blocking its function in activating the subsequent signaling or events in the complement cascade. An antibody or antibody therapeutic of the present application can be a full-length immunoglobulin, a monoclonal antibody, a chimeric antibody (e.g., a humanized antibody), a single chain antibody, a domain antibody, an Fab fragment, or an antibody having an Fab fragment and a mutated Fc portion.

In some embodiments, the disclosure relates to administration of therapeutically effective amounts of the CFP inhibitor and/or CFB inhibitor, e.g., anti-CFP antibody and/or anti-CFB antibody. A “therapeutically effective amount” as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject. Methods for determining therapeutically effective amount of the inhibitory molecules of the disclosure, including, compositions comprising such molecules, have been described in the Examples. For e.g., in subjects having or suspected of having BA, therapeutically effective amounts of the CFP inhibitors and/or CFB inhibitors may confer qualitative or quantitative benefit in one or more of the following parameters in vivo: (a) prevention of acute growth failure; (b) weight gain; (c) improved recovery from jaundice; and/or (d) prevention of early mortality.

The term “modulate,” “modulates,” or “modulation” refers to enhancement (e.g., an increase) or inhibition (e.g., a decrease) in the specified level or activity. As used herein, the terms “enhance,” “ameliorate” or “increase” refer to an increase in the specified parameter (e.g., weight) of at least about 15%, 25%, 35%, 40%, 50%, 60%, 75%, 80%, 90%, 95% or more, e.g., 2-fold, 3-fold, 4-fold, 5-fold, 6-fold, 8-fold, or 10-fold. As used herein, the terms “inhibit,” “attenuate” or “reduce” refer to a decrease or diminishment in the specified parameter of at least about 15%, 25%, 35%, 40%, 50%, 60%, 75%, 80%, 90%, 95% or more.

The inhibitors, e.g., anti-CFP antibody and/or anti-CFB antibody, of the present disclosure may be administered to any subject, e.g., human subject or an animal, by any means known in the art. The present disclosure relates to both veterinary and medical applications. Suitable subjects include both avians and mammals. Non-limiting examples include pigs, cows, horses, goats, sheep, llamas and alpacas; a companion animal such as dogs, cats, rabbits, and birds; a zoo animal such as non-human primates, large cats, wolves, and bears, etc. Human subjects include neonates, infants, juveniles, and adults.

In one aspect, the individual may be one having a disease characterized by liver fibrosis. In one aspect, the disease may be biliary atresia. In one aspect, the individual may be an individual who has undergone a procedure for biliary atresia (Kasai Procedures). The method may comprise administering to the individual in need thereof a therapeutically effective amount of one or both of a Complement Factor B inhibitor and a Complement Factor P (“CFP” or “properdin”) inhibitor. In a further aspect, the individual may further be administered N-acetyl cysteine. This may be administered before, after, or concurrently with the administration of the one or both of a Complement Factor B inhibitor and a Complement Factor P (“CFP” or “properdin”) inhibitor.

In one aspect, the method may include administering one or both of a Complement Factor B inhibitor and a Complement Factor P (“CFP” or “properdin”) inhibitor in an amount sufficient to attenuate and/or reverse biliary fibrosis, for example, biliary-atresia associated hepatobiliary injury, one or more of intrahepatic and/or hepatobiliary inflammation, intrahepatic and/or hepatobiliary fibrosis, cholangiopathy, periductal inflammation, fibrosis, ballooning degeneration, confluent necrosis, portal inflammation, lobular inflammation, bile duct injury, bile duct fibrosis, portal and and/or pericellular bile duct fibrosis, and combinations thereof, in an individual in need thereof, particularly a pediatric subject, more particularly an individual less than three months of age.

In one aspect, the biliary atresia may be characterized by one or more symptoms selected from jaundice, pruritis, cirrhosis, hypercholemia, neonatal respiratory distress syndrome, lung pneumonia, increased serum concentration of bile acids, increased hepatic concentration of bile acids, increased serum concentration of bilirubin, hepatocellular injury, liver scarring, liver failure, hepatomegaly, xanthomas, malabsorption, splenomegaly, diarrhea, pancreatitis, hepatocellular necrosis, giant cell formation, hepatocellular carcinoma, gastrointestinal bleeding, portal hypertension, hearing loss, fatigue, loss of appetite, anorexia, peculiar smell, dark urine, light stools, steatorrhea, failure to thrive, and/or renal failure.

In one aspect, the individual being treated may be a pediatric subject. In one aspect, the pediatric patient may be a newborn, a pre-term newborn, an infant, a toddler, a preschooler, a school-age child, a pre-pubescent child, post-pubescent child, an adolescent, or a teenager under the age of eighteen. In some embodiments, the pediatric patient is a newborn, a pre-term newborn, an infant, a toddler, a preschooler, or a school-age child. In some embodiments, the pediatric patient is a newborn, a pre-term newborn, an infant, a toddler, or a preschooler. In some embodiments, the pediatric patient is a newborn, a pre-term newborn, an infant, or a toddler. In some embodiments, the pediatric patient is a newborn, a pre-term newborn, or an infant. In some embodiments, the pediatric patient is a newborn. In some embodiments, the pediatric patient is an infant. In some embodiments, the pediatric patient is a toddler.

In one aspect, the administration of the disclosed compositions may be carried out in an amount and until attenuation and/or reversal of a symptom of liver disease occurs. For example, the administration may be carried out until a symptom of biliary atresia or post-Kasai Procedure biliary atresia is improved. Such symptom may be one or more of, for example intrahepatic and/or hepatobiliary inflammation, intrahepatic and/or hepatobiliary fibrosis, cholangiopathy, periductal inflammation, fibrosis, ballooning degeneration, confluent necrosis, portal inflammation, lobular inflammation, bile duct injury, bile duct fibrosis, portal and and/or pericellular bile duct fibrosis.

In one aspect, the administration of the disclosed compositions may promote a regenerative response in a liver and/or a bile duct cell in an individual having a liver disease characterized by fibrosis, such as, for example, biliary atresia. This response may be achieved by administration of one or both of a Complement Factor B inhibitor and a Complement Factor P (“CFP” or “properdin”) inhibitor. The administration may occur at a time point selected from pre-, post-, or during a Kasai Procedure.

In one aspect, the administration of the disclosed compositions may preserve, restore, or improve liver function in an individual having a liver disease characterized by fibrosis, such as, for example, biliary atresia. This response may be achieved by administration of one or both of a Complement Factor B inhibitor and a Complement Factor P (“CFP” or “properdin”) inhibitor at a time point selected from pre-, post-, or during a Kasai Procedure. In one aspect, the administration may reduce or eliminate the need for a liver transplant in an individual having a liver disease characterized by fibrosis, such as an individual having biliary atresia or post-Kasai Procedure biliary atresia.

The timing of administering a therapeutic to an individual can vary, for example, depending on the identity of the subject or the cholestatic liver disease or condition to be treated or prevented, or both. For example, the administration may occur before the manifestation of biliary atresia, during the manifestation of biliary atresia, or after the manifestation of biliary atresia. In other aspects, the administration may occur before a Kasai Procedure, during a Kasai Procedure, or after a Kasai Procedure.

Where the administration occurs in conjunction with a Kasai procedure, for example, wherein a first dose is administered during the procedure, immediately following the procedure, within 12 hours of the procedure, within 24 hours of the procedure, or within 48 hours of the procedure, or within 72 hours of the procedure, or within 96 hours of the procedure. A second dose may be administered at a second time point, for example, approximately 1 to about 30 days, or from about 2 to about 20 days, or about from about 3 to about 10 days following the procedure. In other aspects, a third dose may be administered. A third dose may be administered at a time point of about 60 to 90 days following the Kasai procedure, or about 70 to 80 days following the Kasai procedure. In other aspects, the dose may be administered daily, or twice a day, for any number of days following the procedure. In one aspect, the one or both of a Complement Factor B inhibitor and a Complement Factor P (“CFP” or “properdin”) inhibitor may be administered until improvement in a phenotypic outcome occurs, for example, a phenotypic outcome such as attenuation or reversal of inflammation and/or fibrosis progression. In one aspect, the one or both of a Complement Factor B inhibitor and a Complement Factor P (“CFP” or “properdin”) inhibitor may be administered in an amount sufficient to reduce serum biomarkers of liver injury selected from ALT, AST and/or Bilirubin.

In one aspect, one or both of a Complement Factor B inhibitor and a Complement Factor P (“CFP” or “properdin”) inhibitor may be administered at a dose of from about 1 mg/kg to about 10 mg/kg. In one aspect, the One or both of a Complement Factor B inhibitor and a Complement Factor P (“CFP” or “properdin”) inhibitor may be administered intravenously and/or subcutaneously.

In some embodiments, the active agents provided herein may be provided to an administering physician or other health care professional in the form of a kit. The kit is a package which houses a container which contains the active agent(s) in a suitable pharmaceutical composition, and instructions for administering the pharmaceutical composition to an individual. The kit may optionally also contain one or more additional therapeutic agents currently employed for treating a disease state as described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of an active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to an individual.

### EXAMPLE 1

### Complement Factor B (CFB) Blocking Studies (Standard/Classical EHBD Injury Model)

Complement Factor B (CFB) blocking studies were carried out using the Standard/Classical model of EHBD injury, obstruction and atresia associated with hepatocellular injury necrosis and portal inflammation. Newborn Balb/c mice were injected i.p. with 1.0×10⁶ ffu of rhesus rotavirus (RRV) within 24 hrs of birth. Normal saline-injected newborn mice were used as controls. Infected mice were treated using anti-CFB Abs (mAb 1379; 40 mg/kg) with first dose given 6 hours after RRV infection and daily until day 14 of life. Experimental mice were sacrificed at day 14 post infection and Ab treatment. EHBDs and livers were microdissected, plasma collected by intracardiac punctures and histopathologies were assessed by standard techniques. CFB is increased in experimental BA correlating to hepatobiliary inflammation and fibrosis. CFB levels in serum of patients with BA show increased CFB levels and stratification into high and low expressors. Serum CFB levels were estimated using high throughput SomaLogic SOMAscan proteomics in BA patients (N=137) and normal subjects (N=7). Increased levels of CFB indicate systemic dysregulation of complement activation and advanced clinical disorder disease. Blocking CFB prevents acute growth failure and promotes weight gain in neonatal mice with experimental BA. Anti-CFB Abs promote recovery from jaundice associated with development of BA in early neonatal life. Treatment with anti-CFB Abs prevents early mortality in >85% of mice with experimental BA. Blocking CFB prevents EHBD inflammation, cholangiocyte injury and progression to duct atresia. Histology of EHBDs after anti-CFB Ab treatments show significant reductions in epithelial injury, periductal inflammation and intra-ductal immune cell infiltrations. Blocking CFB attenuates ductal edema, mucosal immune cell infiltrations and epithelial disruptions. anti-CFB Ab treatment prevents portal inflammation, loss of hepatocytes and necroinflammatory patches. Blocking CFB attenuates liver portal inflammation and necroinflammatory changes characteristic of neonatal BA. anti-CFB Ab treatment suppressed reactive bile duct proliferation, a poor prognostic factor in patients with BA. Blocking CFB attenuates intrahepatic bile duct proliferations in neonatal mice with experimental BA.

### EXAMPLE 2

### Complement Factor B (CFB) Blocking Studies (Liver Fibrosis Model)

Complement Factor B (CFB) blocking studies were carried out using the Liver Fibrosis model of intrahepatic bile duct injury, bridging fibrosis and portal inflammation replicative of progressive hepatobiliary injury of human BA. Neonatal Balb/c mice were injected i.p. with 1.8×10⁶ ffu of rhesus rotavirus (RRV) on day 3 of life. Normal saline injected newborn mice were used as controls. Infected mice were treated using anti-CFB Abs (mAb1379; 40 mg/kg) with first dose given 6 hours after RRV infection and daily until day 22 of life. Experimental mice were sacrificed at day 19 post infection and Ab treatment. EHBDs and livers were microdissected, plasma collected by intracardiac punctures and histopathologies were assessed by standard techniques. Blocking CFB substantially decreased immune cell infiltrations, portal inflammation and hepatocellular necrosis. Blocking CFB attenuates portal inflammation, liver injury and necrosis and intrahepatic cholangitis. Treatment with anti-CFB Abs significantly reduced portal biliary fibrosis in infantile mice with liver fibrosis. Blocking CFB attenuates biliary fibrosis and portal expansions characteristic of progressive BA. anti-CFB Abs substantially suppressed bile duct proliferations associated with progressive hepatobiliary disease. Blocking CFB attenuates intrahepatic bile duct proliferations in infantile mice with BA-associated liver fibrosis.

### EXAMPLE 3

### Complement Factor P (Properdin) Blocking Studies

RNASeq analysis of livers from patients with BA identifies decreased expression of the inhibitory factor CFI, CFH, and CD45. CFI is a potent inactivator of C3b and plays an essential role in regulating and inhibiting the immune response by controlling all complement pathways. Essential co-factors include CFH, CD46 (cell surface) and C4BP (fluidphase). CFH acts as a soluble inhibitor of complement and accelerates the decay of the complement alternative pathway (AP) C3 convertase C3bBb, thus preventing local formation and amplification of additional C3b. CD46 acts as a cofactor for CFI and protects autologous cells against C-mediated injury by cleaving C3b and C4b deposited on host tissue. CD46 also acts as a costimulatory factor for T-cells which induces the differentiation of CD4+ into T-regs. Livers from patients with BA also show significantly low expressions of C4BPA and C4BPB. RNASeq analysis of livers from patients with BA identifies increased expression of the activating factor CFD. CFD cleaves CFB as its only substrate when complexed to C3b and is the rate-limiting step of AP of complement activation. Increased levels are found in heart failure patients with advanced clinical disease. RNASeq analysis also identified differential expressions of key complement components C9, CD59 and CFHR5.

### EXAMPLE 4

### Complement Factor P (Properdin) Blocking Studies (EHBD Model)

Complement Factor P (CFP) blocking studies were carried out using the Standard/Classical model of EHBD injury, obstruction and atresia associated with hepatocellular injury necrosis and portal inflammation. Newborn Balb/c mice were injected i.p. with 1.0×10⁶ ffu of rhesus rotavirus (RRV) within 24 hrs of birth. Normal saline-injected newborn mice were used as controls. Infected mice were treated using anti-CFP Abs (mAb 1379; 40 mg/kg) with first dose given 6 hours after RRV infection and every 4th day until day 14 of life. Experimental mice were sacrificed at day 14 post infection and Ab treatment. EHBDs and livers were microdissected, plasma collected by intracardiac punctures and histopathologies were assessed by standard techniques. CFP is increased in experimental BA correlating to hepatobiliary inflammation and fibrosis. CFP levels in serum of patients with BA: Consumption of CFP indicates activation of AP. Serum CFP levels were estimated using highthroughput SomaLogic SOMAscan proteomics in BA patients (N=137) and normal subjects (N=7). Decreased levels of CFP are associated with advanced clinical disorder and clear manifestations of the disease due to consumption. CFP expressions in livers of patients with BA show similar patterns: decreased levels and increased consumption. Blocking CFP prevents acute growth failure and promotes weight gain in neonatal mice with experimental BA. Anti-CFP Abs promote recovery from jaundice associated with development of BA in early neonatal life. Treatment with anti-CFP Abs prevents early mortality in >85% of mice with experimental BA. Blocking CFP prevents EHBD inflammation, cholangiocyte injury and importantly progression to duct atresia. Histology of EHBDs after anti-CFP Ab treatments show significant reductions in epithelial injury, periductal inflammation and intra-ductal immune cell infiltrations. Blocking CFP attenuates ductal edema, mucosal immune cell infiltrations and epithelial disruptions. anti-CFP Ab treatment prevents portal inflammation, loss of hepatocytes and necroinflammatory patches. Blocking CFP attenuates liver portal inflammation and necroinflammatory changes characteristic of neonatal BA. anti-CFP Ab treatment suppressed reactive bile duct proliferation, a poor prognostic factor in patients with BA. Blocking CFP attenuates intrahepatic bile duct proliferations in neonatal mice with experimental BA.

### EXAMPLE 5

### Complement Factor P (Properdin) Blocking Studies (Liver Fibrosis Model)

Complement Factor P (CFP) blocking studies using the liver Fibrosis model of intrahepatic bile duct injury, bridging fibrosis and portal inflammation replicative of progressive hepatobiliary injury of human BA were carried out. Neonatal Balb/c mice were injected i.p. with 1.8×10⁶ ffu of rhesus rotavirus (RRV) on day 3 of life. Normal saline injected newborn mice were used as controls. Infected mice were treated using anti-CFP Abs (mAb 1379; 40 mg/kg) with first dose given 6 hours after RRV infection and every 4th day until day 22 of life. Experimental mice were sacrificed at day 19 post infection and Ab treatment. EHBDs and livers were microdissected, plasma collected by intracardiac punctures and histopathologies were assessed by standard techniques. Blocking CFP substantially decreased immune cell infiltrations, portal inflammation and hepatocellular necrosis. Blocking CFP attenuates portal inflammation, liver injury and necrosis and intrahepatic cholangitis. Treatment with alpha-CFP Abs significantly reduced portal biliary fibrosis in infantile mice with liver fibrosis. Blocking CFP attenuates biliary fibrosis and portal expansions characteristic of progressive BA. anti-CFP Abs substantially suppressed bile duct proliferations associated with progressive hepatobiliary disease. Blocking CFP attenuates intrahepatic bile duct proliferations in infantile mice with BA-associated liver fibrosis.

Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1. A composition comprising an inhibitor selected from a Complement Factor B inhibitor, a Complement Factor P (“CFP” or “properdin”) inhibitor, or a combination thereof for use in treating a disease characterized by or leading to biliary fibrosis in an individual, optionally wherein said inhibitor is a humanized antibody or fragment or derivative thereof.

Example 2. The composition of Example 1, wherein said inhibitor is selected from an anti-CFB antibody or fragment thereof, an anti-CFP antibody or fragment thereof, a humanized anti-CFB antibody or fragment thereof, a humanized anti-CFP antibody or fragment thereof, and combinations thereof.

Example 3. The composition of Examples 1 or 2, wherein said inhibitor is selected from a monoclonal anti-CFB antibody or fragment thereof, a monoclonal anti-CFP antibody or fragment thereof, a humanized monoclonal anti-CFB antibody or fragment thereof, a humanized monoclonal anti-CFP antibody or fragment thereof, and combinations thereof.

Example 4. The composition of any of Examples 1, 2, or 3, wherein said inhibitor is an antigen-binding antibody fragment, wherein said antigen-binding fragment inhibits one or both of CFB or CFP

Example 5. The composition of any of Examples 1 through 4, wherein said inhibitor is an anti-CFB antibody or fragment thereof, wherein said antibody or fragment thereof is human, humanized, chimerized, or deimmunized.

Example 6. The composition of any of Examples 1 through 4, wherein said inhibitor is an anti-CFP antibody or fragment thereof, wherein said antibody or fragment thereof is human, humanized, chimerized, or deimmunized.

Example 7. The composition of any of Examples 1 through 6, wherein said inhibitor is a human, humanized, chimerized, or deimmunized antibody or antibody fragment.

Example 8. The composition of any of Examples 1 through 7, wherein said disease is cholestatic liver disease.

Example 9. The composition of any of Examples 1 through 8, wherein said disease is biliary atresia.

Example 10. The composition of Example 9, wherein said biliary atresia is post-Kasai biliary atresia.

Example 11. The composition of any of Examples 1 through 10, wherein said individual is a human subject.

Example 12. The composition of any of Examples 1 through 11, wherein said individual is a pediatric subject.

Example 13. The composition of any of Examples 1 through 12, wherein said composition attenuates and/or reverses liver fibrosis.

Example 14. The composition of any of Examples 1 through 13, wherein said composition attenuates and/or reverses one or more of biliary-atresia associated hepatobiliary injury, intrahepatic and/or hepatobiliary inflammation, intrahepatic and/or hepatobiliary fibrosis, cholangiopathy, periductal inflammation, fibrosis, ballooning degeneration, confluent necrosis, portal inflammation, lobular inflammation, bile duct injury, bile duct fibrosis, portal and and/or pericellular bile duct fibrosis, and combinations thereof, in said subject.

Example 15. The composition of any of Examples 1 through 14, wherein said composition promotes a regenerative response in a liver and/or a bile duct cell in said subject.

Example 16. The composition of any of Examples 1 through 15, wherein said composition preserves, restores, or improves liver function in said subject.

Example 17. The composition of any of Examples 1 through 16, wherein said composition reduces the need for a liver transplant in said subject.

Example 18. The composition of any of Examples 1 through 17, wherein said composition is used after said individual has undergone a Kasai procedure, optionally wherein a first dose is administered at a time point selected from at the time of said procedure, within about 1 to 72 hours of said procedure, or within about 8 to 36 hours of said procedure, or within about 48 hours of said procedure.

Example 19. The composition of any of Examples 1 through 18, wherein said one or both of said Complement Factor B inhibitor and said Complement Factor P (“CFP” or “properdin”) inhibitor improves or substantially normalizes a serum biomarkers of liver injury selected from one or more of conjugated bilirubin (Bc), aspartate aminotransferase (AST), alanine aminotransferase (ALT), gamma-glutamyltransferase (GGT), albumin, sodium, total bilirubin (TB), platelets, international normalized ratio (INR), 25-hydroxy Vitamin D, Vitamin A, and Vitamin E.

Example 20. The composition of any of Examples 1 through 19, wherein said one or both of said Complement Factor B inhibitor and said Complement Factor P (“CFP” or “properdin”) inhibitor achieves one or more parameters selected from normalization or improvement of Total serum bile acids (TSBAs), an improvement or normalization of weight gain, an improvement or normalization of total bilirubin concentration; an improvement or normalization of weight height; an improvement or normalization of ascites, an improvement or normalization of bile drainage, an improvement or normalization of circulating Tregs (CD4+CD25+FoxP3+), CD3/4 T cells, CD3/8 T cells, NK cells (CD56), NK T cells (CD3/56), CD19/20 B cells, macrophages (CD14/11b), and neutrophils, an improvement or normalization of plasma levels of anti-enolase antibody, an improvement or normalization of plasma cytokine levels (Th1/Th2 multiplex and IL17), and improvement or reversal of inflammation and/or fibrosis progression.

Example 21. The composition of any of Examples 1 through 20, wherein said one or both of said Complement Factor B inhibitor and said Complement Factor P (“CFP” or “properdin”) inhibitor is in a form suitable for administration via a route selected from intravenously, subcutaneously, or a combination thereof.

Example 22. The composition of any of Examples 1 through 21, wherein said inhibitor is administered daily, or at least every other day, or at least twice a week, or at least weekly, or at least bi-weekly, or at least once a month.

Example 23. The composition of any one of Examples 1 through 22, wherein said composition further comprises N-acetyl cysteine.

Example 24. A method for treating a disease characterized by biliary fibrosis, comprising administering a therapeutically effective amount of an inhibitor selected from a Complement Factor B (“CFB”) inhibitor, a Complement Factor P (“CFP” or “properdin”) inhibitor, or a combination thereof to in an individual having said disease characterized by biliary fibrosis.

Example 25. The method of Example 24, wherein said inhibitor is selected from an anti-CFB antibody or fragment thereof, an anti-CFP antibody or fragment thereof, a humanized anti-CFB antibody or fragment thereof, a humanized anti-CFP antibody or fragment thereof, and combinations thereof.

Example 26. The method of Example 24 or 25, wherein said inhibitor is selected from a monoclonal anti-CFB antibody or fragment thereof, a monoclonal anti-CFP antibody or fragment thereof, a humanized monoclonal anti-CFB antibody or fragment thereof, a humanized monoclonal anti-CFP antibody or fragment thereof, and combinations thereof.

Example 27. The method of any of Examples 24 through 26, wherein said inhibitor is an antigen-binding antibody fragment, wherein said antigen-binding fragment inhibits one or both of CFB or CFP.

Example 28. The method of any of Examples 24 through 27, wherein said inhibitor is an anti-CFB antibody or fragment thereof, wherein said antibody or fragment thereof is human, humanized, chimerized, or deimmunized.

Example 29. The method of any of Examples 24 through 27, wherein said inhibitor is an anti-CFP antibody or fragment thereof, wherein said antibody or fragment thereof is human, humanized, chimerized, or deimmunized.

Example 30. The method of any of Examples 24 through 29, wherein said inhibitor is a human, humanized, chimerized, or deimmunized antibody or antibody fragment.

Example 31. The method of any of Examples 24 through 30, wherein said disease is cholestatic liver disease.

Example 32. The method of any of Examples 24 through 31, wherein said disease is biliary atresia.

Example 33. The method of any of Examples 24 through 32, wherein said biliary atresia is post-Kasai biliary atresia.

Example 34. The method of any of Examples 24 through 33, wherein said individual is a human subject.

Example 35. The method of any of Examples 24 through 34, wherein said individual is a pediatric subject.

Example 36. The method of any of Examples 24 through 35, wherein said administration is administered in an amount and for a period of time sufficient to attenuate and/or reverse liver fibrosis.

Example 37. The method of any of Examples 24 through 36, wherein said administration is administered in an amount and for a period of time sufficient to attenuate and/or reverse one or more of biliary-atresia associated hepatobiliary injury, intrahepatic and/or hepatobiliary inflammation, intrahepatic and/or hepatobiliary fibrosis, cholangiopathy, periductal inflammation, fibrosis, ballooning degeneration, confluent necrosis, portal inflammation, lobular inflammation, bile duct injury, bile duct fibrosis, portal and and/or pericellular bile duct fibrosis, and combinations thereof, in said subject.

Example 38. The method of any of Examples 24 through 37, wherein said administration promotes a regenerative response in a liver and/or a bile duct cell in said subject.

Example 39. The method of any of Examples 24 through 38, wherein said administration preserves, restores, or improves liver function in said subject.

Example 40. The method of any of Examples 24 through 39, wherein said administration reduces the need for a liver transplant in said subject.

Example 41. The method of any of Examples 24 through 40, wherein said administration occurs after said individual has undergone a Kasai procedure, for example, wherein a first dose is administered at a time point selected from at the time of said procedure, within about 1 to 72 hours of said procedure, or within about 8 to 36 hours of said procedure, or within about 48 hours of said procedure.

Example 42. The method of any of Examples 24 through 41, wherein said one or both of said Complement Factor B inhibitor and said Complement Factor P (“CFP” or “properdin”) inhibitor is administered in an amount sufficient to improve or substantially normalize a serum biomarkers of liver injury selected from one or more of conjugated bilirubin (Bc), aspartate aminotransferase (AST), alanine aminotransferase (ALT), gamma-glutamyltransferase (GGT), albumin, sodium, total bilirubin (TB), platelets, international normalized ratio (INR), 25-hydroxy Vitamin D, Vitamin A, and Vitamin E.

Example 43. The method of any of Examples 24 through 42, wherein said one or both of said Complement Factor B inhibitor and said Complement Factor P (“CFP” or “properdin”) inhibitor is administered until an outcome of one or more parameters is achieved: normalization or improvement of Total serum bile acids (TSBAs), an improvement or normalization of weight gain, an improvement or normalization of total bilirubin concentration; an improvement or normalization of weight height; an improvement or normalization of ascites, an improvement or normalization of bile drainage, an improvement or normalization of circulating Tregs (CD4+CD25+FoxP3+), CD3/4 T cells, CD3/8 T cells, NK cells (CD56), NK T cells (CD3/56), CD19/20 B cells, macrophages (CD14/11b), and neutrophils, an improvement or normalization of plasma levels of anti-enolase antibody, an improvement or normalization of plasma cytokine levels (Th1/Th2 multiplex and IL17), and improvement or reversal of inflammation and/or fibrosis progression.

Example 44. The method of any of Examples 24 through 43, wherein said one or both of said Complement Factor B inhibitor and said Complement Factor P (“CFP” or “properdin”) inhibitor is administered via a route selected from intravenously, subcutaneously, or a combination thereof.

Example 45. The method of any of Examples 24 through 44, wherein said inhibitor is administered daily, or at least every other day, or at least twice a week, or at least weekly, or at least bi-weekly, or at least once a month.

Example 46. The method of any of Examples 24 through 45, further comprising administering N-acetyl cysteine.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1-23. (canceled)

24. A method for treating a disease characterized by biliary fibrosis, comprising administering a therapeutically effective amount of an inhibitor selected from a Complement Factor B (“CFB”) inhibitor, a Complement Factor P (“CFP” or “properdin”) inhibitor, or a combination thereof, to in an individual having said disease characterized by biliary fibrosis.

25. The method of claim 24, wherein said inhibitor is selected from an anti-CFB antibody or fragment thereof, an anti-CFP antibody or fragment thereof, a humanized anti-CFB antibody or fragment thereof, a humanized anti-CFP antibody or fragment thereof, and combinations thereof.

26. The method of claim 24, wherein said inhibitor is selected from a monoclonal anti-CFB antibody or fragment thereof, a monoclonal anti-CFP antibody or fragment thereof, a humanized monoclonal anti-CFB antibody or fragment thereof, a humanized monoclonal anti-CFP antibody or fragment thereof, and combinations thereof.

27. The method of claim 24, wherein said inhibitor is an antigen-binding antibody fragment, wherein said antigen-binding fragment inhibits one or both of CFB or CFP.

28. The method of claim 24, wherein said inhibitor is an anti-CFB antibody or fragment thereof, wherein said anti-CFB antibody or fragment thereof is human, humanized, chimerized, or deimmunized.

29. The method of claim 24, wherein said inhibitor is an anti-CFP antibody or fragment thereof, wherein said anti-CFP antibody or fragment thereof is human, humanized, chimerized, or deimmunized.

30. The method of claim 24, wherein said inhibitor is a human, humanized, chimerized, or deimmunized antibody or antibody fragment.

31. The method of claim 24, wherein said disease is cholestatic liver disease.

32. The method of claim 24, wherein said disease is biliary atresia.

33. The method of claim 32, wherein said biliary atresia is post-Kasai biliary atresia.

34. The method of claim 24, wherein said individual is a human subject.

35. The method of claim 24, wherein said individual is a pediatric subject.

36. The method of claim 24, wherein said administration is administered in an amount and for a period of time sufficient to attenuate and/or reverse liver fibrosis.

37. The method of claim 24, wherein said administration is administered in an amount and for a period of time sufficient to attenuate and/or reverse one or more of biliary-atresia associated hepatobiliary injury, intrahepatic and/or hepatobiliary inflammation, intrahepatic and/or hepatobiliary fibrosis, cholangiopathy, periductal inflammation, fibrosis, ballooning degeneration, confluent necrosis, portal inflammation, lobular inflammation, bile duct injury, bile duct fibrosis, portal and and/or pericellular bile duct fibrosis, and combinations thereof, in said individual.

38. The method of claim 24, wherein said administration promotes a regenerative response in a liver and/or a bile duct cell in said individual.

39. The method of claim 24, wherein said administration preserves, restores, or improves liver function in said individual.

40. The method of claim 24, wherein said administration reduces a need for a liver transplant in said individual.

41. The method of claim 24, wherein said administration occurs after said individual has undergone a Kasai procedure, optionally wherein a first dose is administered at a time point selected from at the time of said procedure, within about 1 to 72 hours of said procedure, or within about 8 to 36 hours of said procedure, or within about 48 hours of said procedure.

42. The method of claim 24, wherein said one or both of said Complement Factor B inhibitor and said Complement Factor P (“CFP” or “properdin”) inhibitor is administered in an amount sufficient to improve or substantially normalize a serum biomarkers of liver injury selected from one or more of conjugated bilirubin (Bc), aspartate aminotransferase (AST), alanine aminotransferase (ALT), gamma-glutamyltransferase (GGT), albumin, sodium, total bilirubin (TB), platelets, international normalized ratio (INR), 25-hydroxy Vitamin D, Vitamin A, and Vitamin E.

43. The method of claim 24, wherein said one or both of said Complement Factor B inhibitor and said Complement Factor P (“CFP” or “properdin”) inhibitor is administered until one or more normalization or improvement of Total serum bile acids (TSBAs), an improvement or normalization of weight gain, an improvement or normalization of total bilirubin concentration; an improvement or normalization of weight height; an improvement or normalization of ascites, an improvement or normalization of bile drainage, an improvement or normalization of circulating Tregs (CD4+CD25+FoxP3+), CD3/4 T cells, CD3/8 T cells, NK cells (CD56), NK T cells (CD3/56), CD19/20 B cells, macrophages (CD14/11b), and neutrophils, an improvement or normalization of plasma levels of anti-enolase antibody, an improvement or normalization of plasma cytokine levels (Th1/Th2 multiplex and IL17), improvement or reversal of inflammation and/or fibrosis progression, and combinations thereof.

44. The method of claim 24, wherein said one or both of said Complement Factor B inhibitor and said Complement Factor P (“CFP” or “properdin”) inhibitor is administered via a route selected from intravenously, subcutaneously, or a combination thereof.

45. The method of claim 24, wherein said inhibitor is administered daily, or at least every other day, or at least twice a week, or at least weekly, or at least bi-weekly, or at least once a month.

46. The method of claim 24, further comprising administering N-acetyl cysteine.
